(19) United States
(12) Patent Application Publication
(10) Pub. No.: US 2008/0267963 A1
(43) Pub. Date: Oct. 30, 2008

(54) Methods of Cancer Treatment/Prevention Using Cancer Cell-Specific Surface Antigens

(75) Inventors: Edward H. Kislauskis, Medway, MA (US); Kejian Yang, Northborough, MA (US); Barbara J. Whalen, Shrewsbury, MA (US)

Correspondence Address: ROPES & GRAY LLP, PATENT DOCKETING 39/41, ONE INTERNATIONAL PLACE, BOSTON, MA, 02110-2624, US

(73) Assignee: Biomedical Research Model, Inc., Worchester, MA (US)

(21) Appl. No.: 11/666,956

(22) PCT Filed: Oct. 27, 2005
(86) PCT No.: PCT/US05/38968

§ 371 Date: May 21, 2008

Related U.S. Application Data
(60) Provisional application No. 60/624,296, filed on Nov. 2, 2004.

(51) Int. Cl. A61K 9/127; A61K 39/00; A61K 39/395; A61P 35/04

(52) U.S. Cl. 424/138.1; 424/450; 424/277.1

(57) ABSTRACT

The invention relates to methods and reagents for immunizing and treating individual with cancer to elicit specific cellular and humoral immune-responses against specific cancer cell surface antigens, including those cancer cell surface antigens expressed only in cancer cells and in non-cancer cells normally located in one or more immune-privileged sites or tissues of the individual. The invention provides methods of using specifically prepared immunogen in fresh or lyophilized liposome, proper routes of administration of the immunogen, proper doses of the immunogen, and specific combinations of heterologous immunization including DNA priming in one administration route followed by liposome-mediated protein antigen boost in a different route to tailor the immune responses in respects of enhancing cell mediated immune response, cytokine secretion, humoral immune response, especially skewing T helper responses to be Th1 or a balanced Th1 and Th2 type.

### REFERENCE TO RELATED APPLICATION

This application claims the benefit of the filing date of U.S. Provisional Application Ser. No. 60/624,296, entitled “METHODS OF CANCER TREATMENT/PREVENTION USING CANCER CELL-SPECIFIC SURFACE ANTIGENS,” and filed on Nov. 2, 2004. The teachings and the entire specification of the referenced application are incorporated herein by reference.

### BACKGROUND OF THE INVENTION

The development of effective vaccines against a variety of agents is an important objective for disease control worldwide. Protective immunity against specific antigen is achieved by humoral, cellular and mucosal immune responses. Humoral or antibody responses are important in pathogen neutralization and can be very effective in some conditions such as cancer and infectious diseases. Antibodies circulate in the blood, and provide a measure of the overall humoral immune responses. Cell mediated immunity also plays an important role in immune surveillance against cancer and many intracellular pathogens.

In many cases, repeated immunization may result in enhanced immune responses and confer increased protection. A priming immunization establishes a base level of immunity and can lead to the development of T and B cell memory. A booster immunization at a later time can mobilize these memory cells and lead to higher and more specific immune responses.

Vaccine delivery takes a variety of forms, depending on the agent to be delivered and the administration route. Vaccine delivery systems are often designed to administer vaccines to specific areas of the body. Immunization may be achieved by oral, intramuscular administration of antigens, such as injection of antigens in the presence of preservatives, adjuvants, and other excipients via intramuscular or subcutaneous routes to elicit protective immunity. Parenteral immunization may also be used.

Therapeutic cancer vaccines (TCVs) are a new class of active specific immunotherapy agents that trigger a targeted immune response against cancer. There is a need for TCVs in addition to those presently available.

### SUMMARY OF THE INVENTION

The present invention relates to a method for eliciting an immune response in an individual in need thereof, against a cancer cell antigen, especially a surface antigen. In certain embodiments, the cancer cell antigen may be intracellular antigens in origin, but which may be released when cancer cells die or become abnormally expressed in cancer cells. In certain embodiments, the method comprises administering to the individual an antigen-liposome preparation, which comprises the cancer cell surface antigen and a liposome, in sufficient dose to elicit an immune response to the cancer cell surface antigen, wherein the immune response is sufficient to treat the cancer in the patient, such as by alleviating at least one symptoms of the cancer, preventing the development and/or progression of the cancer, and/or improving the life quality of the cancer patient.

In certain embodiments, the cancer cell surface antigen is expressed only in cancer cells in the individual.

In certain embodiments, the cancer cell surface antigen is expressed only by (a) cancer cells, and (b) non-cancer cells normally located in one or more immune-privileged sites or tissues in the individual. The one or more immune-privileged sites of the individual are, for example, brain, spinal cord, eye (e.g., anterior chamber, vitreous cavity, subretinal space, etc.), adrenal cortex, and a reproductive organ (e.g., testis, ovary, uterus—especially pregnant uterus, etc.).

In certain embodiments, the cancer cell surface antigen is a neuropeptide (such as pro-vasopressin), a neuroendocrine peptide, or a neuroendocrine peptide receptor.

In certain embodiments, the cancer cells are from organs with endocrine function, such as kidney (adrenal gland), stomach, pancrease, thyroid and parathyroid glands, thymus, ovary, and testis, etc.

In certain embodiments, the cancer cells are from small cell lung cancer, breast cancer, or prostate cancer, or neuroendocrine cancer.

In certain embodiments, the neuroendocrine tumor is a carcinoid tumor; an adrenal pheochromocytoma; a gastrinoma (causing Zollinger-Ellison syndrome); a glucagonoma; an insulinoma; a medullary carcinoma of the thyroid; a multiple endocrine neoplasia syndrome; a pancreatic endocrine tumor; a paraganglioma; a VIPoma (vasoactive intestinal polypeptide tumor).

In certain embodiments, the carcinoid tumor is found in gastrointestinal tract, lung, intestinal tract, appendix, rectum, bronchial tubes, or ovary.

In certain embodiments, the method and composition are useful for inducing an immune response to a cell surface antigen for treating metastatic breast cancer. In this embodiment, the antigen is, for example, a mutant glycosylation of (cancer cell) mucin that results in expression or exposure of cancer associated epitopes (e.g., STn). Although mucins are found on both normal epithelial cells and cancer cells, mutant glycosylation is evident on cancer cell surfaces but not on normal cell surfaces. In one embodiment, the antigen is a dissacharide antigenic epitope that mimics variant mucin molecules present on cancer cells (e.g. STn, Biomira USA Inc., Cranbury, N.J.).

In another embodiment, the antigen is, for example, a peptide-based antigen BLP25 that elicits an immune response to a particular MUC-1 mucin that is present on solid tumors. The BLP25 vaccine is useful for treating, for example, metastatic non-small cell lung cancer.

Any other cancer cell surface antigens may be used in the present invention. Such cancer antigens include, for example, abnormal version of a normal protein that contains novel epitopes (e.g., abnormal but novel carbohydrate such as the STn vaccine, supra; or newly exposed wild-type polypeptide fragments that are not accessible in normal cells because these peptides are buried deep underneath the normal mucin carbohydrate chains).

In another embodiment, the antigen is, for example, gangliosides such as GD3. Such antigens are found on the surfaces of certain types of tumors (e.g., melanomas, small cell lung cancer [SCLC]). In certain embodiments, if the gangliosides are poorly immunogenic, peptide mimetics (such as anti-idiopathic antibody) resembling such gangliosides may be used. For example, BEC2 is a monoclonal anti-idiopathic antibody mimicking GD3. BEC2 exerts a stronger immunogenic effect than the original GD3 antigen. SCLC patients developed anti-BEC2 antibodies against the original tumor antigen GD3. Currently, BEC2 is in a phase III trial for SCLC-limited disease in the U.S., Europe, Australia and New Zealand.

In another embodiment, there is provided a method and compositions for inducing an immune response to a cell surface antigen for treating cancer (such as hepatocellular carcinoma) in a mammal. In this embodiment, the cancer cell surface antigen is at least a part of an alpha fetoprotein (AFP) molecule expressed at the cell surface. Such antigens are described in, for example, US20030143237A1, which is incorporated herein by reference.

In addition to providing protein antigens, DNA encoding sucg protein antigens may be provided as a DNA vaccine. In certain embodiments, the DNA vaccine is administered orally. In certain embodiments, the DNA vaccine is administered as a liposome composition, such as the ones described herein. For example, the cancer antigen may be a cell surface antigen such as vascular endothelial growth factor receptor (e.g. VEGFR2), which is preferentially found in tumor vasculature (Niethammer et al. Nature Medicine 8(12): 1369-1375, 2002). In one embodiment, the antigen is administered, for example, orally, in the form of DNA vaccine. Since tumor progression and growth depends, to a large extent, on angiogenesis, immunotherapy directed against proliferating endothelial cells can be used to selectively target malignancy, including pre-existing malignancy.

One advantage of this DNA approach, as an anti-angiogenesis therapy, is that it obviates the need to administer large amounts of anti-angiogenesis compound to kill endothelial cells. Instead, it relies on the fact that immune response depends on T cells and/or B cells, which also have memory and could slow down or even prevent a recurring disease. Any other tumor vessel-specific proteins can be such a vaccine.

The method and compositions can also be used to induce an immune response to a cell surface antigen for treating cancer, such as small cell lung cancer. In this embodiment, the cancer cell surface antigen is the glycopeptide region (MAG-1) of provasopressin (pro-VP). See Keegan et al. (Molecular Cancer Therapeutics 1: 1153-1159, 2002). The pro-VP protein is a protein normally found at an immune-privileged location (i.e. hypothalamic neurons of the brain). This protein is normally packaged into secretory vesicles, where it undergoes enzymatic cleavage to generate VP, VP-NP, and VAG (North, In: D. Gash and G. Boer (eds.), Vasopressin: Principles and Properties, pp. 175-209. New York: Plenum Press, 1987). These components are then secreted into the circulation. SCLC tumors and cultured cells also express the VP gene. Intact pro-VP protein can become localized to the cell surface plasma membrane (Friedmann et al., Br. J. Cancer 69: 260-263, 1994; North et al., Ann. N.Y. Acad. Sci. 689: 107-121, 1993). Polyclonal antibodies raised against VP-NP bind specifically to the surface of cultured SCLC cells, as determined by immuno-fluorescence analysis (Friedmann et al., Neuropeptides 28: 183-189, 1995; North et al., Prog. Brain Res. 60: 217-225, 1983; North and Yu, Regul. Pept. 45: 209-216, 1993). The pro-VP protein may be used as an antigen for inducing an immune response according to the method of the present invention. In one embodiment, the antigen is a synthetic peptide representing the COOH-terminal portion of the VAG region of the pro-VP protein.

Thus the cell surface antigen may be, for example, alpha fetal protein (AFP), BLP25 vaccine or MUC1 mucin, STn or STn-KLH, GD3 or its anti-idiotypic antibody BEC2, and VEGFR2.

The individual treated by the present method can be any mammal, such as a human or a non-human animal.

In certain embodiments, the antigen-liposome preparation is freshly prepared.

In certain embodiments, the antigen-liposome preparation is lyophilized before use.

In certain embodiments, the antigen-liposome preparation is administered intranasally, intramuscularly, parenterally, subcutaneously, intravenously, or orally to the individual.

In certain embodiments, the antigen-liposome preparation is administered with at least one vaccine adjuvant, such as LT R192G.

In certain embodiments, the method further comprises measuring humoral and/or cellular immune responses to the cancer cell surface antigen.

In certain embodiments, the humoral immune response includes total antibody (Ig) titers in serum or at mucosal surfaces; titers of anti-HBsAg-specific antibodies in serum or at mucosal surfaces; titers of specific antibody isotypes and/or sub-types including IgG, IgA, IgG1, and IgG2a; ratio of IgG1 and IgG2a.

In certain embodiments, the cellular immune response includes in vivo cytotoxic T cell (CTL) activity; secretion of cytokines characteristic of Th1 response including IL-12 and IFN-gamma; secretion of cytokines characteristic of Th2 response including IL-4, IL-5, IL-10, and IL-13; and T-helper cell profile (e.g., Th1 versus Th2 response).

In certain embodiments, the humoral and/or cellular immune responses are measured from samples obtained from the mammalian individual about 2, 4, 6, or 8 weeks post the last boost, or about 8 weeks post the initial administration.

This invention also relates to a method for selectively killing cancer cells expressing a cancer cell surface antigen in an individual in need thereof, comprising administering to the individual an antigen-liposome preparation, under conditions that result in production in the individual of antibodies against the cancer cell surface antigen, wherein the antibodies produced bind the cancer cell surface antigen on cancer cells in the individual, and the cancer cells that express the cancer cell surface antigen are killed.

In certain embodiments, the cancer cell surface antigen is expressed only in cancer cells in the individual.

In certain embodiments, the cancer cell surface antigen is expressed only by (a) cancer cells, and (b) non-cancer cells normally located in one or more immune-privileged sites or tissues in the individual. The one or more immune-privileged sites of the individual are, for example, brain, spinal cord, eye (e.g., anterior chamber, vitreous cavity, subretinal space, etc.), adrenal cortex, and a reproductive organ (e.g., testis, ovary, uterus—especially pregnant uterus, etc.).

In certain embodiments, the cancer cell surface antigen is a neuropeptide (such as pro-vasopressin), a neuroendocrine peptide, or a neuroendocrine peptide receptor; while the cancer cells are small cell lung cancer cells, breast cancer cells, or prostate cancer cells.

Another aspect of the invention is a method for selectively killing cancer cells expressing a cancer cell surface antigen in an individual in need thereof, comprising administering to the individual a therapeutically effective amount of the cancer cell surface antigen, under conditions that result in production of antibodies against the cancer cell surface antigen, wherein the cancer cell surface antigen is expressed only in cancer cells and in non-cancer cells normally located in one or more immune-privileged sites or tissues of the individual, and the antibodies bind the cancer cell surface antigen on cancer cells in the individual.

In certain embodiments, the one or more immune-privileged sites of the individual are selected from the group consisting of: brain, spinal cord, anterior chamber of the eye, vitreous cavity and subretinal space of the eye, adrenal cortex, and a reproductive organ selected from the group consisting of: testis, ovary, and uterus—especially pregnant uterus.

In certain embodiments, the cancer cell surface antigen is a neuropeptide (such as pro-vasopressin), a neuroendocrine peptide, or a neuroendocrine peptide receptor, and the cancer cells are small cell lung cancer cells, breast cancer cells, or prostate cancer cells.

In certain embodiments, the cancer cell surface antigen is in a liposome preparation.

Another aspect of the invention provides a method of treating a cancer in an individual in need thereof, while protecting non-cancer cells of the individual from the adverse effects of the treatment, comprising administering to the individual a therapeutically effective amount of a composition comprising a cancer cell surface antigen, under conditions that result in production of antibodies against the cancer cell surface antigen, wherein the antibodies bind the cancer cell surface antigen on cancer cells in the individual, and wherein the cancer cell surface antigen is expressed only in cancer cells and in non-cancer cells normally located in one or more immune-privileged sites or tissues of the individual.

In certain embodiments, the one or more immune-privileged sites of the individual are selected from the group consisting of: brain, spinal cord, anterior chamber of the eye, vitreous cavity and subretinal space of the eye, adrenal cortex, and a reproductive organ selected from the group consisting of: testis, ovary, and uterus—especially pregnant uterus.

In certain embodiments, the cancer cell surface antigen is a neuropeptide (such as pro-vasopressin), a neuroendocrine peptide, or a neuroendocrine peptide receptor, and the cancer cells are small cell lung cancer cells, breast cancer cells, or prostate cancer cells.

In certain embodiments, the cancer cell surface antigen is in a liposome preparation.

In certain embodiments, where appropriate, the liposome preparation is prepared from a composition comprising Phosphatidylcholine/cholesterol/dicetyl Phosphate in 7/3/0.5 mole %.

In certain embodiments, the average size of liposomes in the liposome preparation is between 0.1 μm to about 5 μm.

In certain embodiments, the average size of a mixed liposome preparation is about 0.2 μm, about 1 μm, or about 24 μm.

Other features and advantages of the invention will be apparent from the following detailed description and claims.

### BRIEF DESCRIPTION OF THE DRAWINGS

FIG. 1 illustrates kinetics of HBsAg-specific serum Ig levels in CD1 mice after intranasal immunization with HBsAg encapsulated in liposomes.

FIG. 2 illustrates HBsAg-liposome dose response in CD1 mice immunized intranasally, 8 weeks after first immunization.

FIG. 3 is an example that shows the effect of route of administration and delivery of a booster immunization on total HBsAg-specific serum antibody in CD1 mice administered HBsAg-liposomes (15 μg/dose). Closed symbols show responses in mice (n=5) that received a single priming immunization. Closed symbols with cross-hatches (+) show responses in mice (n=5) that received identical primary and secondary booster immunizations.

FIG. 4 is an example that shows the effect of route of administration and delivery of a booster immunization on total HBsAg-specific serum antibody in Balb/c mice administered HBsAg-liposomes (15 μg/dose). Closed symbols show responses in mice (n=5) that received a single priming immunization. Closed symbols with cross-hatches (+) show responses in mice (n=5) that received identical primary and secondary booster immunizations.

FIG. 5 is an example that shows the total serum Ig level 8 weeks post oral immunization in CD1 mice, with or without second boost, with freshly prepared or lyophilized HBsAg-liposome.

FIG. 6 is an example that shows the HBsAg-specific serum Ig level 8 weeks post intranasal (IN) immunization in CD1 mice, with or without second boost, with freshly prepared or lyophilized HBsAg-liposome.

FIG. 7 is an example that shows the HBsAg-specific serum Ig level 8 weeks post intramuscular (IM) immunization in CD1 mice, with or without second boost, with freshly prepared or lyophilized HBsAg-liposome. The data show that lyophilized and reconstituted HBsAg-liposomes retain their immunogenicity. Equal doses of fresh liposomes or lyophilized and reconstituted liposomes (15 μg HBsAg/dose) were delivered IM or IN to CD 1 mice (n=5). Total HBsAg-specific serum antibody responses were measured in the sera 8 weeks after the primary immunization.

FIG. 8 is an example that shows the total serum Ig level 8 weeks post oral or intranasal (IN) immunization in CD1 mice with freshly prepared HBsAg-liposome, either at the presence or absence of LT adjuvant. The mucosal adjuvant LT enhances HBsAg-specific antibody responses when co-administered with HBsAg-liposomes delivered by the intranasal but not the oral route. LT was given at 5 μg/dose. HBsAg-liposomes were given at 15 μg of HBsAg per dose (n=5).

FIG. 9 illustrates a typical result of IgG1:IgG2a in the serum of CD1 mice immunized by different protocols. Grey bars are the IgG1:IgG2a ratio from mice that received one immunization. Black bars are the IgG1:IgG2a ratio from mice that received two immunization—a primary followed by a secondary 6 weeks later. Quantitative HBsAg-specific ELISA assays were used to determine the level of HBsAg-specific IgG1 and IgG2a in the serum from a serum pool (equal amounts of serum from 4-5 individual mice per treatment group). Panel A: Sera from CD1 mice 8 weeks after primary immunization (n=5).

FIG. 10 illustrates a typical result of IgG1:IgG2a in the serum of Balb/c mice immunized by different protocols. Ratio of HBsAg-specific IgG1:IgG2a in the sera of HBsAg immunized Balb/c mice 8 weeks after primary immunization (n=5). Grey bars are the IgG1:IgG2a ratios from mice that received one immunization. Black bars are these ratios from mice that received two immunizations at weeks 0 and 6. The level of HBsAg-specific IgG1 and IgG2a was determined on serum pools using quantitative ELISA assays. Two vertical lines at 0.5 and at 2.0 in each panel demarcate three different patterns of antibody responses. A ratio of 0.5 or less indicates a Th1 polarized response. A ratio of 2.0 or more indicates a Th2 polarized response. Ratios between 0.5 and 2.0 indicate a mixed, or neutral, response.

FIG. 11 is an example that shows the level of total HBsAg-specific serum Ig 8 weeks after immunization, illustrating that antibody responses to HBsAg-DNA vaccine is weak or moderate. HBsAg DNA (Aldevron) was injected IM into the quadriceps muscle (50 μl/muscle) of CD1 or Balb/c mice (n=5) at week 0 or both at week 0 and at week 6. Antibody responses were measured at 8 weeks.

FIG. 12 illustrates the effect of heterologous immunization is Balb/c mice: Immunization with HBsAg-DNA intramuscularly (2× at 100 μg each) generates specific T cell memory that is revealed with a low dose of HBsAg-liposomes (3 μg/dose, n=8 mice per group). Total HBsAg-specific serum antibody was measured four weeks after the IN boost.

FIG. 13 illustrates the effect of low dose IN boost with HBsAg-liposomes (3 μg) on IgA responses in HBsAg-DNA primed Balb/c mice. Priming Balb/c mice with HBsAg-DNA IM (2× at 100 μg/dose) generates memory for serum and mucosal secretory IgA responses which is revealed following an intranasal boost with HBsAg-liposomes. HBsAg-DNA was injected IM at weeks 0 and 6. HBsAg-liposomes (3 μg/dose) were delivered intranasally on week 10. HBsAg-specific IgA responses were measured 4 weeks after the boost (n=8).

FIG. 14 is an example that shows IgG1:IgG2a ratios following a heterologous immunization protocol with HBsAg-DNA priming and low dose HBsAg-liposome boost.

FIG. 15 illustrates the effect of vaccine adjuvant LT R192G on Th type polarization in Balb/c and CD1 mice immunized and boosted with HBsAg-liposomes IN. The effect is measured 8 weeks post immunization. Co-administration of the mucosal adjuvant LT with HBsAg-liposomes shifts the immune response from a Th2 or mixed response to a Th1-biased response. LT (5. μg/dose) and HBsAg-liposomes (15 μg HBsAg/dose) were given IN to mice (n=5) on weeks 0 and 6. Quantitative ELISA assays were used to determine serum levels of IgG1 and IgG2a two weeks after the second immunization.

FIG. 16 is representative of the degree of variation that is observed between individual CD1 and Balb/c mice within an experiment. The top panel shows HBsAg-specific serum antibody from individual CD1 mice. Responses are shown from mice that received one (closed circles) or two (closed triangles) immunizations. End-point titers were defined as a serum titer >2× that observed for naïve mice. Horizontal lines denote the average response. Similarly, data from Balb/c mice are depicted in the bottom panel.

FIG. 17 is an example showing that co-administration of the mucosal adjuvant LT with HBsAg-liposomes enhances HBsAg-specific serum and secretory IgA responses. LT (5 μg/dose) and HBsAg-liposomes (15 μg HBsAg/dose) were given IN to mice (n=5) on weeks 0 and 6. HBsAg-specific IgA ELISA assays were used to determine IgA levels in the serum and vaginal washes.

FIG. 18 is an example that shows effect of liposome size on serum HBsAg-specific IgG responses in Balb/c mice (6 weeks after primary immunization and two-weeks post boost). Liposomes containing HBsAg (15 μm) were sized at 4 μm, 1 μm and 0.2 μm and were administered intranasally to Balb/c mice (n=5) as a single size, or as an equal mixture of all three sizes. The vertical bar in the mix group shows the predicted value if the antibody responses are additive.

FIG. 19 top panel shows spleen cells from naïve Balb/c mice, and the bottom panel shows spleen cells from Balb/c mice that received the heterologous immunization regimen. Region 1 is gated on non-fluorescing (recipient) spleen cells. Region 2 is gated on CFSE^{low} (nonspecific peptide pulsed donor) cells. Region 3 is gated on CFSE^{high} (HBsAg-peptide pulsed donor) cells. 200,000 viable cells were analyzed. The reduction in the number of cells in region 3 in the right panel demonstrates HBsAg-specific killing of the transfused target cells.

### DETAILED DESCRIPTION OF THE INVENTION

### I. Overview

One aspect of the instant invention provides a method for eliciting an immune response against a cancer cell surface antigen in an individual in need thereof. The method comprises administering to the individual an antigen-liposome preparation in sufficient dose to elicit the immune response to the cancer cell surface antigen, wherein the antigen-liposome preparation comprises the cancer cell surface antigen and a liposome.

In certain embodiments, the cancer cell surface antigen is expressed only in cancer cells in the individual, such that the immune response specific for the antigen would not attack healthy tissues/organs of the individual. In certain embodiments, the antigen that is expressed on the cancer cell surface is a mutant protein present only on cancer cells, and the immune response is specific for the mutant protein epitope comprising the mutation. The mutation can be a protein mutation, or abnormal sugar or lipid structures on cancer cell surface.

Alternatively, the cancer cell-specific antigen is a normal, non-mutant protein that is usually (normally) only expressed in a previous developmental stage (e.g. fetus, etc.) of the individual. The cancer cell antigen might be “specific” to the cancer cell, in the sense that it is a “normal” protein without amino acid sequence mutation, but such protein in a normal individual is typically subject to one or more post-translational modifications (e.g. phosphorylation, proteolytic cleavage, etc.), while in cancer cells, such post-translational modifications are lacking.

In some other embodiments, the cancer cell surface antigen may be expressed both in cancer cells, and in noncancerous (normal) cells of the individual, but due to the location of the normal cells, they are not subject to attacks by the immune system of the individual. For example, these tissues or organs might be located in so-called immune privileged sites in mammals. Immune-privileged sites include, but are not limited to: anterior chamber of the eye, vitreous cavity and subretinal space of the eye, CNS (e.g. brain and spinal cord), uterus—especially pregnant uterus, ovary, testis, and adrenal cortex, etc.

If an antigen is expressed only on cancer cell surfaces and on cells in one or more of these immune privileged locations/tissues, immunization as described herein can be used to immunize the individual in need of cancer treatment, since the resulting immune response would be rather specific for the cancer cells, with minimal, if any collateral damage to other normal tissues.

Cancer cell surface antigens, particularly those cancer cell surface antigens expressed only by (a) cancer cells, and (b) non-cancer cells normally located in one or more immune-privileged sites or tissues in the individual, may be identified, either from literature or through screening, for use in the instant immunization method. Such cancer antigens may include: altered mucin carbohydrates and/or exposed mucin peptide fragments, partial alpha-fetal proteins (e.g. those disclosed in US 2003/0143237, etc.).

In certain embodiments, the cancers mis-express and/or overexpress certain neuroendocrine peptides or their receptors. Many such cancers express the neuroendocrine peptides or their receptors on cancer cell surface. Since neuroendocrine peptides are typically secreted by cells in the brain (an immune previleged site), normal cells are not susceptible to immune system attack if such neuroendocrine peptides are used as vaccines/antigens in the subject immune-therapeutic methods. One typical such cancer include various neuroendocrine tumors occurring at various organs/tissues.

Neuroendocrine tumor refers to the cell type and/or origin and/or distribution in the body of neuroendocrine cells. Neuroendocrine cells produce hormones or regulatory proteins, and so tumors of these cells usually have symptoms that are related to the specific hormones that they produce. Neuroendocrine cells have roles both in the endocrine system and the nervous system. They produce and secrete a variety of regulatory hormones, or neuropeptides, which include neurotransmitters and growth factors. When these cells become cancerous, they grow and overproduce their specific neuropeptide. Thus, in addition to behaving similarly to typical cancers, neuroendocrine tumors cause the body to produce a detrimentally excessive amount of hormones, possibly leading to asthma, cardiac disease, dehydration and profuse diarrhea.

Neuroendocrine tumors are generally rare. The most common types of neuroendocrine tumors found in adults are carcinoid and oat cell. While neuroendocrine tumors can originate anywhere in the body, carcinoid tumors are most commonly found in the gastrointestinal tract or lungs. Other types of neuroendocrine tumors are: adrenal pheochromocytomas; gastrinomas (causing Zollinger-Ellison syndrome); glucagonomas; insulinomas; medullary carcinomas of the thyroid; multiple endocrine neoplasia syndromes; pancreatic endocrine tumors; paragangliomas; VIPomas (vasoactive intestinal polypeptide tumor). Carcinoid tumor can occur in the intestinal tract, appendix, rectum, bronchial tubes, or ovary. Most carcinoid tumors secrete serotonin. When the blood concentration of this hormone is high enough, it causes carcinoid syndrome. This syndrome refers to a variety of symptoms that are caused by the excessive amount of hormone secreted rather than the tumor itself.

Neuroendocrine tumors are typically slow-growing tumors produce non-specific symptoms, making diagnosis a challenge. But if left untreated, these tumors can be life threatening. On the other hand, however, with early detection and careful monitoring, this condition can be controlled. At present, the only treatment for carcinoid tumor is surgical removal of the tumor, which of course depends on the correct diagnosis of such tumors. Chemotherapy is sometimes used when metastasis has occurred, it is rarely effective, and the 5-year survival rate for patients with metastatic neuroendocrine tumors is less than 20%. In contrast, the subject immune-therapeutic methods are uniquely suitable for treating this type of tumor, since vaccines are typically used to prevent the occurrence and/or progression of diseases, even when there is no detectable symptoms yet.

The method of the invention can be used in any individual with immune response, including but not limited to mammals. The mammal may be a human, or a non-human animal. The animal may be, for example, a domestic livestock animal, such as cow, sheep, goat, pig, horse; a pet, such as cat, dog, rabbit or other rodents; or a laboratory animal such as mouse, rat, hamster, guinea pig, etc.

The invention provides that cancer cell surface antigens as vaccines can be incorporated into liposome preparations with a defined composition/size. Such antigen-liposome preparation can be delivered directly to a specific location of the individual, such as mucosal surfaces. In addition, by combining such antigen-liposome vaccines with appropriate adjuvants and immunization regimens, immune responses could be tailored to provide more effective vaccines.

Most immune responses are regulated by T lymphocytes, which initiate and shape the nature of the response. As immune responses nature, CD4⁺ T lymphocytes can become polarized towards T helper type 1 (Th1) or T helper type 2 (Th2) immune responses. The hallmark of Th1 and Th2-type responses is the predominant pattern of cytokines that are present. Th1 responses are characterized by high levels of IFN-γ and low levels of IL-4, while Th2 responses are characterized by low levels of IFN-γ and high levels of IL-4. These cytokines play an important role in determining the functional capabilities of the T cells. Th2-type responses lead to the preferred production of antibodies of the IgG1 subclass, with little or no generation of CTLs. Th1-type responses lead to the preferred production of antibodies of the IgG2a subclass and induction of CTLs that can effectively kill cells infected with viruses or other organisms.

In certain embodiments, immunization results in a balanced or T helper 1 (Th1)-biased immune response that also includes robust antibody responses, CTL generation and Th1-type cytokine production, and may include local immunity at mucosal sites. In other embodiments, it may be possible to tailor the immune response to generate a T helper 2 (Th2)-biased immune response.

As used herein, “T helper type 1 response” and “Th1 response” are used interchangeably to refer to a range of host animal responses including one or more, usually all the characteristics listed in the middle column of Table A above. These characteristics include a ratio of IgG1:IgG2a of no greater than 0.5; increased IFN-gamma and IL-12 (and other Th1 cytokines) secretion by T helper 1 cells and decreased IL-4 (and other Th2 cytokines) secretion by T helper 2 cells; and high CTL activity.

Similarly, as used herein, “T helper type 2 response” and “Th2 response” are used interchangeably to refer to a range of host animal responses including one or more, usually all the characteristics listed in the right column of Table A above. These characteristics include a ration of IgG1:IgG2a of no less than 2.0; decreased IFN-gamma and IL-12 (and other Th1 cytolines) secretion by T helper 1 cells and increased IL-4 (and other Th2 cytokines) secretion by T helper 2 cells; and low or absent CTL activity.

Aluminum (alum) is the only adjuvant that is FDA approved for use in human vaccines. When co-administered with protein-based vaccines, it induces a serum antibody response but no mucosal immunity or cytolytic T lymphocyte (CTL) responses. Alum-based vaccines therefore generate immune responses that are strongly biased towards a T helper 2-type (Th2) cytokine response, which may provoke the development of allergic responses to environmental antigens. In contrast, DNA vaccines delivered intramuscularly can promote CTL responses and Th1-type cytokine responses but are generally weak inducers of antibody responses. Neither alum-based protein vaccines nor DNA vaccines elicit good immune responses locally at mucosal surfaces.

The instant invention provides methods to determine the optimal conditions for the incorporation of various antigens (such as various cancer cell surface antigens) into liposomes (e.g., dose of cancer cell surface antigen, liposome size, liposome: protein ratio, and fresh versus lyophilized and reconstituted liposomes) to generate the most robust antibody responses. Serum antibody responses were compared in mice that received such encapsulated antigens in liposomes (cancer cell surface antigen-liposome) by oral, intranasal (IN), and intramuscular (IM) routes of delivery. The invention also discloses the effect of serum antibody response after one administration (rime) or two administrations (prime and boost) of antigen. Liposomes were found to be both a very effective adjuvant and delivery vehicle for inducing immunity to such phathogen antigens as HBsAg, via the intranasal or intramuscular routes.

In one embodiment, antibody responses for the test antigen HBsAg achieved or exceeded the levels observed in mice treated with the commercially available HBsAg GSK vaccine currently used in humans. Targeted delivery of antigen to the mucosal surfaces of the nasal passages also stimulated the local production of secretory IgA in mucosal secretions at all mucosal surfaces sampled. Furthermore, the mucosal vaccine adjuvant LT R192G was successfully co-administered with HBsAg-liposomes to enhance overall antibody levels, direct the immune response towards a Th1-type response, and significantly enhance the production of mucosal IgA. The general success of these immunization methods indicates that similar methods may be used to immunize animals with other antigens, such as the subject cancer cell surface antigens.

The invention additionally provides that heterologous immunization regimens including priming and boosting with different forms of vaccines (e.g., Antigen-DNA IM and Antigen-liposomes IN or M) could result in more robust immune responses with preservation of local immunity at mucosal sites. In one embodiment, for example, priming with HBsAg-DNA vaccine intramuscularly and boosting with HBsAg-liposomes intranasally induced potent synergistic antibody responses, activated CTLs and Th1-type cytokine profiles, and preserved secretory IgA production at mucosal sites. Delivery of the boost by the intranasal route was established as an important determinant for the generation of local immunity.

However, under certain other circumstances, a Th2-type immune response may be preferred. Chen et al. (Transplantation 61(7): 1076-83, 1996) investigated the role of Th1 and Th2 cytokines in rejection and tolerance using the neonatal tolerance model. It was found that lymph nodes that drained immunogen-bearing tolerant grafts produced a 10- to 100-fold higher ratio of interleukin (IL)-4 to interferon (IFN)-γ (a Th2-type response) compared with lymph node cells from rejected grafts. Moreover, because neonatal antigen exposure triggers allospecific Th2 CD4 memory cells, whereas antigen exposure during adulthood triggers Th1 CD4 memory cells, it was speculated that immunoredirection toward Th2 and away from Th1 functions as another mechanism of tolerance. To test this immunoredirection hypothesis, Chen et al. examined whether recovery of Th1 cytokine responses abrogates tolerance, and showed that treatment with exogenous IFN-γ at the time of neonatal priming recovered mixed lymphocyte reaction hypoproliferation and restored the ability of mice to reject skin grafts. Mice that received IFN-γ at the time of neonatal priming produced more IFN-γ and contained more A/J-reactive IFN-γ producing CD4 cells compared with untreated neonatal primed mice, but failed to recover A/J-specific INF-γ-producing CD8 cells or CTL responses, which suggests that graft rejection occurred via Th1 CD4 cells. Interestingly, draining lymph node cells from rejected grafts in IFN-γ-treated neonatal primed mice also produced more IL-4, compared with cells from healthy grafts on untreated neonatal primed mice. Nonetheless, lower IL-4 to IFN-γ ratio predicted graft rejection and higher ratios predicted acceptance. Thus it appears that neonatal tolerance depends on the ability to block generation of allospecific Th1 responses that lead to rejection, and that immunoredirection involves both the inhibition of Th1 and expansion of Th2 immune responses. However, Coudert et al. (J. Clin. Invest. 105: 1125-1132, 2000) also showed that an uncontrolled Th2 response may lead to subsequent lethal autoimmune disease. Therefore, a Th2-biased response (without complete diminished Th1 response) may be beneficial under certain circumstances.

Thus the invention provides several vaccine delivery protocols which promote different types of immune responses (antibody production, T cell cytokine profiles, cellular immunity by CTLs, and local immunity at mucosal surfaces). Rational combinations of these delivery platforms allow for the development of vaccines that are tailored to provide the best protection against specific pathogens under specific circumstances, for example, to favor Th1 or Th2 type immune responses, or a mixed/more balanced response.

In one aspect, the present invention provides methods and reagents for effectively eliciting immune responses in animals, especially mammals, against certain antigens, such as cancer cell surface antigens. Certain embodiments of the invention relates to the use of liposome-protein antigens delivered intranasally (IN) or intramuscularly (IM) to the host animal.

In certain embodiments, the instant invention provides methods and reagents for generating more effective vaccines by incorporating antigens into liposomes of a defined composition and size, and by delivering the resulting antigen-liposome preparation directly to the mucosal immune system. In other embodiments, application of the specific platform delivery technologies of the invention provides rational combination of such antigen-liposome vaccines with appropriate adjuvants and immunization regimens, to tailor immune responses so as to provide more effective and specific vaccines (enhancing cell mediated and humoral immune response, tailoring Th2 and mixed/balanced Th1/Th2 to Th1 response, increasing IFN-γ secretion, induction of high level mucosal IgA, IgG and CTL).

Protective mucosal immune responses are crucial in controlling diseases caused by many naturally occurring infectious organisms and microorganisms that may be used in bioterrorism. Cell mediated immune responses including CTLs are also important in controlling intracellular pathogens both systemically and at mucosal sites. Traditional intramuscularly delivered protein vaccines, which use alum as an adjuvant, generate a typical Th2 response with mainly IgG1 antibody responses, but fail to stimulate CTL responses or local immunity at mucosal sites.

The instant invention provides a liposome based antigen delivery platform technology for vaccine applications via mucosal sites. The invention combines a liposome incorporation technology, specific delivery to mucosal sites, and the use of adjuvants and heterologous immunization protocols to achieve a more balanced or mixed Th response.

Liposomes have several potential advantages as delivery platforms for vaccines. Incorporation of antigens into liposomes sequesters these antigens, thus the liposomes serve as an antigen depot capable of sustained antigen release. In addition, liposomes are biocompatible and biodegradable, and low in toxicity and immunogenicity. When appropriately sized (e.g., >0.1 μm up to 5 μm, may be a mixture of different sizes), liposomes are selectively taken up by antigen-presenting cells in the body, and have the potential to induce both humoral antibody and CTL responses. Liposomes serve as antigen carrier/vehicle as well as being an adjuvant that can be administered repeatedly.

Liposomes of various sizes can be prepared using proprietary methodology as described below. The resulting liposomes, depending on specific preparation protocols, are typically sized at 4 μm, 1 μm, or 0.2 μm by passing preparations through a microfluidizer. Briefly, Liposomes were prepared at the following lipid concentrations: Phosphatidylcholine/cholesterol/dicetyl phosphate 7/3/0.5 mole %. Antigen, such as the subject cancer cell surface antigen, is incorporated into multilaminar liposomes at several concentrations for the testing of immune responses. In one experiment, an average of about 60% of a test antigen (HBsAg) was incorporated into the liposomes. Liposome size was also measured with a N4 MC Submicron Particle Size Analyzer (Coulter Electronics). A representative profile of the 4 μm sized liposomes is shown below. In one experiment, the liposome: protein ratio was decreased to ½ and ⅓ of the content usually used to encapsulate 15 μg of HBsAg. In certain embodiments, antigen preparations can be lyophilized for storage and then reconstituted before use. Both size and potential (zeta, a measure of charge) might be measured before use. The ζ-potential was determined using Zeta-Puls ζ-potential analyzer (Brookhaven Instruments). The lipid: antigen protein ratio can be varied in some preparations in order to determine the importance of this ratio on immune responses to the specific antigen.

Animal immunization protocols are well-known in the art. See, for example, Using Antibodies: A Laboratory Manual, Eds. Harlow and Lane, Cold Spring Harbor Laboratory Press, New York, 1999.

In certain embodiments, the host animal/individual is immunized intranasally (IN) by the subject antigen-liposome preparation to specifically elicit systemic and mucosal IgA response.

In certain embodiments, the host animal/individual is immunized intramuscularly (IM), parenterally, subcutaneously, intravenously, and/or orally by the subject antigen-liposome preparation.

In other embodiments, the host animal/individual is prime immunized by a DNA vaccine (intramuscularly), followed by intranasal boost with a low dose antigen-liposome preparation of the subject invention. The lose dose antigen-liposome preparation, when administered alone to the host animal, is insufficient to elicit a detectable immune response.

The instant invention can be used to effectively immunize host animals against a range of antigens, especially the subject cancer cell surface antigens.

The antigen-liposome preparation of the invention contains liposomes having an average diameter of about 4 microns, although liposomes of 0.2-8 microns may also be useful. The liposome preparation is preferably freshly prepared just prior to use. However, for intranasal and intramuscular administration, lyophilized liposomes can also be used with comparable effectiveness.

In certain embodiments, one or more boost administration may be used after the initial administration. The boost administration can be done 2-8 weeks, preferably 4-6 weeks apart from the previous administration (initial or a prior boost). Typically, one boost administered 4-6 weeks after the initial administration is sufficient.

The boost administration and the initial administration may use the same or different amounts of antigen-liposome preparations, and the boost and the initial administrations can be administered via the same or different routes.

In certain embodiments, LT adjuvants may be used to further enhance the immune response.

The effects of immune response in the host animal can be assessed by various assays, including humoral and cellular immune responses. Humoral immune response includes total antibody (Ig) titers in serum or at mucosal surfaces; titers of HBsAg-specific antibodies in serum or at mucosal surfaces; titers of specific antibody isotypes and/or sub-types including IgG, IgA, IgG1, and IgG2a; ratio of IgG1 and IgG2a. Cellular immune response includes cytotoxic T cell (CTL) activity; antigen-specific proliferation of CD4⁺ and/or CD8⁺ T cells; secretion of cytokines characteristic of Th1 response including IL-12 and IFN-gamma; secretion of cytokines characteristic of Th2 response including IL-4, IL-5, IL-10, and IL-13; detected by ELISPOT assays and T-helper cell profile (e.g., Th1 versus Th2 response).

In certain embodiments, the humoral and/or cellular immune responses are measured from samples obtained from the mammalian individual about 2, 4, 6, or 8 weeks post the last boost, or about 8 weeks post the initial administration.

Another aspect of the invention provide a method for selectively killing cancer cells expressing a cancer cell surface antigen in an individual in need thereof, comprising administering to the individual an antigen-liposome preparation, under conditions that result in production in the individual of antibodies against the cancer cell surface antigen, wherein the antibodies produced bind the cancer cell surface antigen on cancer cells in the individual, thereby killing cancer cells that express the cancer cell surface antigen.

Similarly, in certain embodiments, the cancer cell surface antigen is expressed only in cancer cells in the individual. In other embodiments, the cancer cell surface antigen is expressed only in cancer cells and in non-cancer cells normally located in one or more immune-privileged sites or tissues in the individual, such as those described above.

In certain embodiments, the cancer cell surface antigen is a neuropeptide (such as pro-vasopressin), a neuroendocrine peptide, or a neuroendocrine peptide receptor, and the cancer cells are small cell lung cancer cells, breast cancer cells, or prostate cancer cells.

Another aspect of the invention provides a method for selectively killing cancer cells expressing a cancer cell surface antigen in an individual in need thereof, comprising administering to the individual a therapeutically effective amount of the cancer cell surface antigen, under conditions that result in production of antibodies against the cancer cell surface antigen, wherein the antibodies bind the cancer cell surface antigen on cancer cells in the individual, and wherein the cancer cell surface antigen is expressed only in cancer cells and in non-cancer cells normally located in one or more immune-privileged sites or tissues of the individual, such as those described above.

In certain embodiments, the cancer cell surface antigen is a neuropeptide (such as pro-vasopressin), a neuroendocrine peptide, or a neuroendocrine peptide receptor, and the cancer cells are small cell lung cancer cells, breast cancer cells, or prostate cancer cells.

In certain embodiments, the cancer cell surface antigen is in a liposome preparation, such as the ones described above.

Another aspect of the invention provides a method of treating a cancer in an individual in need thereof, while protecting non-cancer cells of the individual from the adverse effects of the treatment, comprising administering to the individual a therapeutically effective amount of a composition comprising a cancer cell surface antigen, under conditions that result in production of antibodies against the cancer cell surface antigen, wherein the antibodies bind the cancer cell surface antigen on cancer cells in the individual, and wherein the cancer cell surface antigen is expressed only in cancer cells and in non-cancer cells normally located in one or more immune-privileged sites or tissues of the individual, such as those ones described above.

In certain embodiments, the cancer cell surface antigen is a neuropeptide (such as pro-vasopressin), a neuroendocrine peptide, or a neuroendocrine peptide receptor, and the cancer cells are small cell lung cancer cells, breast cancer cells, or prostate cancer cells.

In certain embodiments, the cancer cell surface antigen is in a liposome preparation, such as the ones described above.

In certain embodiments, the subject liposome preparation is prepared from a composition comprising Phosphatidylcholine/cholesterol/dicetyl Phosphate in 7/3/0.5 mole %. The average size of liposomes in the liposome preparation may vary. For example, the size may be between 0.1 μm to about 5 μm. The liposome preparation may also has mixed sizes of liposomes. For example, the average size of a mixed liposome preparation may be about 0.2 μm, about 1 μm, or about 2-4 μm.

### II. Liposome Preparation

Liposomes are structures consisting of a membrane bilayer composed of phospholipids of biological or synthetic origin, usually spherical in shape. Liposomes form naturally when phospholipids or lipids contact water. The structure of liposomes can be manipulated by methods to form them in the laboratory, including the input of energy in the form of heat, sonic energy, freeze-thaw cycles, or shear forces. Because liposomes have features of biological membranes, they can be engineered in the laboratory to contain a variety of biologically and therapeutic relevant complex molecules, including proteins. While not wishing to be bound by any particular theory, it is contemplated that the phospholipid bilayer membrane of liposomes separates and protects entrapped materials in the inner aqueous core from the outside, although it is not impossible that certain compounds may not be fully entrapped within the inner core (e.g. merely associate with the liposome). Both water-soluble and -insoluble substances can be entrapped in different compartments, the aqueous core and bilayer membrane, respectively, of the same liposome. Chemical and physical interaction of these substances can be eliminated because the substances are in these different compartments. Lyophilized Liposomes can be stored for a longer term at room temperature.

There are numerous liposome preparations and improvements thereof that may be used with the subject cancer cell surface antigens. For example, to name but a few, EP0896816A1, EP0267050A2, EP0190926B1, WO04078121A2, EP0938298A1, WO0100247A1, WO0182892A2, WO02064116A2, WO03028701A2, EP0664116B1, WO03002142A1, WO02089771A1, etc. describe various methods of preparing and using liposome preparations for delivering molecules to cells. All are incorporated herein by reference.

In some embodiments of the invention, the liposome preparation can be prepared according to the following methods, or modification thereof.

A. Basic Method for Liposomes with a Size Range of 2-4 μm.

Liposomes of the subject invention were prepared at the following lipid concentrations: Phosphatidylcholine/cholesterol/dicetyl phosphate 7/3/0.5 mole %. Dicetyl phosphate was dissolved in chloroform plus 5% of ethanol, sonicated and phosphatidylcholine and cholesterol were then added. Lipids were dried in a Labconco rotary evaporator for one hour and traces of chloroform were removed by freeze-drying with a Freezone 4.5 Freeze Dry System overnight. The lipid film was hydrated with antigen, such as Hepatitis B surface antigen (HBsAg), at a concentration of 125-300 μg/ml in 10 mM HEPES-buffer, 150 mM NaCl, pH 7.4(HBS), and filtered with a 0.2 μm nylon filter. The mixture was vortexed thoroughly and allowed to sit for 1 hour and then vortexed again to ensure the formation of multilamellar vesicles. The resultant liposomes were then subjected to three cycles of freeze-and-thaw (1 cycle=freezing for one hour and thawing for one hour at room temperature). The size of the liposomes was measured with a N4 MD Submicron Particle Size Analyzer (Coulter Electronics). The ζ-potential was measured using Zeta-Puls ζ-potential analyzer (Brookhaven Instruments) in 5 mM HEPES buffer, 1.0 mM NaCl, pH 7.4.

B. Liposomes with a Size of 1.0 μm

Liposomes of the subject invention were prepared at the following lipid concentrations: Phosphatidylcholine/cholesterol/dicetyl phosphate 7/3/0.5 mole %. Dicetyl phosphate was dissolved in chloroform plus 5% of ethanol, sonicated and phosphatidylcholine and cholesterol were then added. Lipids were dried in a Labconco rotary evaporator for one hour and traces of chloroform were removed by freeze-drying with a Freezone 4.5 Freeze Dry System overnight. The lipid film was hydrated with antigen (e.g., HBsAg) at a concentration of 125-300 μg/ml in 10 mM HEPES-buffer, 150 mM NaCl, pH 7.4, filtered with 0.2μ nylon filter. The mixture was vortexed thoroughly and allowed to sit for 1 hour and then vortexed again to ensure the formation of multilamellar vesicles. The liposomes were then subjected to three cycles of freeze-and-thaw (1 cycle=freezing for one hour and thawing for one hour at room temperature). After the third cycle the liposomes were warmed in water bath to 50° C. and extruded through a polycarbonate filter with a pore size of 1.0 μm using hand-held Avanti-micro extruder. The size of the liposomes was measured as before.

C. Liposomes with a Size of 0.200 μm.

Liposomes of the subject invention were prepared at the following lipid concentrations: Phosphatidylcholine/cholesterol/dicetyl phosphate 7/3/0.5 mole %. Dicetyl phosphate was dissolved in chloroform plus 5% of ethanol, sonicated and phosphatidylcholine and cholesterol were then added. Lipids were dried in a Labconco rotary evaporator for one hour and traces of chloroform were removed by freeze-drying with a Freezone 4.5 Freeze Dry System overnight. The lipid film was hydrated with antigen (e.g., HBsAg) at a concentration of 125-300 μg/ml in 10 mM HEPES-buffer, 150 mM NaCl, pH 7.4, filtered with 0.2 μm nylon filter. The mixture was vortexed thoroughly and allowed to sit for 1 hour and then vortexed again to ensure the formation of multilamellar vesicles. The liposomes were then subjected to three cycles of freeze-and-thaw (1 cycle=freezing for one hour and thawing for one hour at room temperature). After the third cycle, liposomes were warmed in a water bath to 50° C. and extruded first through a polycarbonate filter with a pore size of 1.0 μm, then with pore size of 0.4 μm and finally 0.2 μm using a hand-held Avanti micro-extruder. Then size of the liposomes was measured as previously.

It is contemplated that the exact size of the liposome in the final preparation may vary over a range to some extent, due to, for example, fusion of some liposomes.

In addition, a mixture of liposomes with different size ranges may be used in the method of the invention.

### D. Preparation of Lyophilized Liposomes.

Liposomes of the subject invention were prepared at the following lipid concentrations: Phosphatidylcholine/cholesterol/dicetyl Phosphate 7/3/0.5 mole %. Dicetyl phosphate was dissolved in chloroform plus 5% of ethanol, sonicated and phosphatidylcholine and cholesterol were then added. Lipids were dried in a Labconco rotary evaporator for one hour and traces of chloroform were removed by freeze-drying with a Freezone 4.5 Freeze Dry System overnight. The lipid film was hydrated with antigen (e.g., HBsAg) at a concentration of 125-300 μg/ml in HBS with 100 mg/ml maltose, filtered with 0.2 μm nylon filter. The mixture was vortexed thoroughly and allowed to sit for 1 hour and then vortexed again to ensure the formation of multilamellar vesicles. The liposomes were then subjected to three cycles of freeze-and-thaw (1 cycle=freezing for one hour and thawing for one hour at room temperature). The size of the liposomes was measured and liposomes were lyophilized with a Freezone 4.5 Freeze Dry System overnight. Lyophilized liposomes were reconstituted by vortexing with corresponding quantity of water to reach the needed antigen concentration and liposome size was measured.

### III. Assays

The following assays may be needed to carry out certain steps of the invention. In general, as a skilled artisan is aware, many different but functionally equivalent assays may be used to achieve the same purpose. Thus none of the specifically described assays are limiting unless explicitly stated.

### Enzyme-Linked Immunosorbent Assay (ELISA)

Anti-HBsAg specific antibody responses such as Ig, IgG, IgA, IgG1 and IgG2a in the collected samples of the immunized mice were tested by ELISAs. A three layer (antigen-antibody-HRP antibody) ELISAs were designed for testing Ig, IgG, IgG1 and IgG2a antibody. 50 μl of 10 μg/ml of pure HBsAg (Advanced Immune Chemical) was coated in a 96 well ELISA plates. Serial dilutions of serum or secretory samples were added after blocking procedure. Goat anti-mouse Ig(H+L)-HRP, goat anti-mouse IgG, goat anti-mouse IgG1 and IgG2a (SouthernBiotech) were used as detecting antibody to test anti-HBsAg specific Ig, IgG, IgG2a and IgG1 in collected samples. Serially diluted IgG, IgG1 and IgG2a standards were used to generate standard curves for IgG, IgG1 and IgG2a antibody. A five layer capture ELISA were used to test mouse IgG. A 4 mg/ml of rat-antimouse IgG was coated to the ELISA plates to capture IgA antibody from serum or mucosal (fecal, vaginal and saliva) secretions. 2 μg/ml of pure HBsAg was added to bind to anti-HBsAg specific IgA antibody. A goat anti-HBsAg is used to specifically bind to the capture HBsAg in the previous step. A Donkey anti-goat IgG HRP was the final detecting antibody before color development from the substrate-TMB.

### T Cell Proliferation Assay

There are numerous ways to assay for T-cell proliferation. The in vitro protocol below is merely one of the possible ways to carry out the assay. Variations of the protocol is well within the scope of the invention.

T-cells can be purified from immunized animal (e.g., mouse) as below:
Pack 0.6 g of pre-washed nylon-wool into a 10 ml syringe, soak with PBS, and autoclave for 20 minutes. Replace the PBS with Dulbecco's Minimum Essential Medium (DMEM) supplemented with 1% homologous normal serum, penicillin-streptomycin (1×), and 2-mercaptoethanol (2-ME-5×10⁻⁵ M). This is called complete DMEM.
Prepare a single cell suspension from spleen or lymph node cells from immunized animals (e.g., mice), wash 3 times with DMEM and then suspend the cells in 1-2 ml of complete DMEM (to reach approximately 1×10⁸ cells/ml).
Load the cell suspension onto the column. Incubate the column for 30 minutes in a CO₂ incubator at 37° C. Elute unbound cells with 20 ml DMEM and then centrifuge at 800 rpm for 8 minutes. Suspend the enriched T-cells at 4-8×10⁶ cells/ml in complete DMEM.

Antigen Presenting Cells (APCs) can be prepared as following:
Use spleen or thymus cells obtained from young, normal congenic animals (6-8 weeks old) as APC Wash the cells with DMEM 3 times and suspend at 1×10⁷ cells/ml in complete DMEM.
Irradiate the cells with 2000 rad=20 Gy. Do not disturb the cells after irradiation.

For T cell proliferation assay:
Add 0.1 ml of enriched T-cells (4-8×10⁶ cells/ml), 0.1 ml of APC (1×10⁷ cells/ml) and 10-20 μl of 1 mg/ml antigen, or a range of antigens starting with 0.1 mM to 20 mM concentration (or positive control, negative control) in a 96-well flat bottom plate. Repeat well set up in triplicate. Culture the cells in a CO₂ incubator at 37° C. for 3 days (or longer if response is small).
Add 0.5-1.0 μCi/well ³H-labeled thymidine (such as NET-027X from NEN-Perkin Elmer) on day 3. The cells are typically suspended with the multichannel to ensure even distribution of the ³H-Thy in the solution. After an additional 15-18 hours of culturing, harvest the cells on a glass filter and assay for the incorporated ³H by scintillation counting. Even if a proliferative response is not seen initially, the cells may have grown up during an in vitro stimulation. To detect smaller proliferation, ³H may be added only on day 4 or 5 to achieve more sensitively.

T cell activation can also be assessed using in vivo assays. In order to evaluate the cell mediated immune responses generated by the subject vaccine platform delivery protocols, in vivo CTL assays were also established to measure cytolytic T lymphocyte (CTL) responses and ELISPOT assays to enumerate frequencies of antigen-specific IFN-γ and IL-4 secreting cells (Th1 and Th2 responses, respectively).

The in vivo CTL assay uses mice that have received two rounds of HBsAg-DNA vaccine IM followed by the subject liposome preparation (e.g., HBsAg-liposome) IN. After the sample collection is completed for the measurement of antibody responses (six weeks or longer after the IN boost), mice receive a DNA boost to mobilize the memory T cells into activated effector cells. 8-12 days later, the activity of mobilized effector cells is quantified by determining their ability to specifically kill HBsAg peptide-pulsed targets that are adoptively transferred intravenously into the mice. The mice receive two populations of cells from naïve mice that are differentially labeled with the intravital dye CFSE (fluorescence emission in the FITC channel): CFSE^{low} cells that are not pulsed with peptide, and CFSE^{high} cells that are pulsed with the L (d) restricted immunodominant peptide from HBsAg (28-39 of HBsAg). The disappearance of the peptide-pulsed CFSE^{high} population relative to the unpulsed CFSE^{low} population is the measure of peptide-specific killing according to the formula shown below. The percentages of these two populations in the cohorts of naïve recipients serves as the internal control for cell engraftment. A summary of this assay is shown below.

### Target Cell Preparation:

Naïve splenocytes: Label with CFSE^{low}. Pulse without peptide. Verify labeling by flow cytometry.
Naïve splenocytes: Label with CFSE^{high}. Pulse with peptide. Verify labeling by flow cytometry.
Mix equal numbers of CFSE^{low} and CFSE^{high} cells (1−1.5×10⁷ cells of each).

### In Vivo CTL Assay:

Transfer cells to cohorts of both immunized and naïve mice IV.
20-24 hours later, harvest spleens and process for flow cytometry and ELISPOT assays.

$$Ratio = Percentage of {CFSE}^{low } cells \text{:} {CFSE}^{high} cells$$

$$\% Specific Lysis = 1 - \underline{ratio in naïve mice} \times 100$$

$$ratio in immunized mice$$

It was found that in vivo CTL activity from heterologously immunized Balb/c mice averaged 78% (range of 65-91% specific killing, N=4). Splenocytes from the immunized mice also had high frequencies of IFNγ but not IL-4 secreting cells in ELISPOT assays after culture with peptide in vitro for 24 hours (data not shown). These results demonstrate that this heterologous immunization protocol does indeed generate robust cell mediated immune responses with a Th1 response biased cytokine profile. These results directly confirm the conclusion derived from the determination of IgG1: IgG2a HBsAg-specific ratios in the serum of the immunized mice. The ELISPOT plates can be further quantitatively analyzed for both frequencies and spot size using a digital plate reader and software designed for this purpose.

In addition, CTL activity can be directly measured at the mucosal surfaces (nasal passages, lung and vagina) by measuring CFSE peaks on frozen tissue sections using digital fluorescence imaging on tissues from the same immunized mice.

### Analysis of Cytokine Secretion by ELISPOT

Spleen cells from immunized mice and naïve were cultured in vitro for the measurement of IFN-γ and IL-4 secreting cells by ELISPOT assays. The ELISPOT assays were established using commercial antibody pairs and reagents (BD Pharmingen) and Multiscreen-IP multiwell ELISPOT plates (Millipore, Hopkington, Mass.). In vitro cultures were stimulated with HBsAg peptide at a concentration of 10 μg/2.5×10⁶ cells per ml for 20-22 hours. Cultures with no peptide were used as a negative control, and cultures stimulated with anti-CD3 or Con A were used as positive controls. High frequencies of HBsAg-specific IFN-γ secreting cells were observed, but only very low frequencies of IL-4 secreting cells were seen. These results directly confirm the conclusion, derived from the analysis of serum IgG1: IgG2a ratios, that the heterologous immunization protocol generates a Th1-type cytokine biased immune response. IFN-γ and IL-4 ELISPOT plates from this study were quantitatively analyzed by Zellnet Consulting Inc. (New York, N.Y.) for both frequencies of spots and spot size using a digital plate reader and software designed for this purpose.

### Analysis of the Cytokine Profile of the Proliferating T Cells

Many commercial kits are available to assay T cell cytokine profiles. For example, the FastImmune Cytokine Detection System (Cat. No. 340971) from BD Immunocytometry Systems (San Jose, Calif.) provides a method for detecting intracellular cytokine production using flow cytometry. The protocol involves fixing and permeabilizing the cells of interest (including T cells, or other cells) and then staining for surface markers and cytokine(s) using fluorescently labeled antibodies. The samples are then analyzed by flow cytometry. This method has many potential uses and can produce useful cytokine profiles from a mixture of cell types including whole blood.

The FastImmune products can be routinely used for analyzing cytokine production in human T cells as well as other human blood cells. In a typical experiment, stimulated T cells are stained with fluorescently conjugated anti-IFN-gamma and/or anti-TNF-alpha to determine antigen specificity for a given human donor. In conjunction with the cytokine antibodies, an antibody cocktail for T cell surface markers including CD69 (a T cell activation marker), CD4 and CD8 may also be used. Using four-color flow cytometry, it is possible to identify specific CD4 and CD8 populations of cytokine producing cells from stimulated T cells. Typically, a range of responses from 0.1% to 1-2% of CD4 or CD8 cells are detected as cytokine positive.

The biggest advantage to this protocol is that it allows the detection of cytokine production from a specific cell type since you can detect surface markers of the producing cells. Using kits that detect a cytokine that is secreted by cells does not identify the phenotype of the active cell. Another advantage is the cytokine positive cells can be sorted using flow cytometry. Therefore, the FastImmune cytokine detection system has the potential to provide valuable information on cytokine production from a mixture of cells such as peripheral blood mononuclear cells, purified T cells, or whole blood.

### Antibody Isotyping

Numerous commercial kits are available to determine antibody isotypes. For example, the Mouse Monoclonal Isotyping Kit (Cat. No. RPN29) by Amersham Biosciences (Piscataway, N.J.) provides a rapid and sensitive means of determining the class, subclass and light chain type of mouse monoclonal antibodies. Typing sticks are exposed to the monoclonal antibody in ascitic fluid or culture supernatant then incubated sequentially with detection reagents supplied in the kit. The identity of the immunoglobulin chains is revealed by the occurrence of purple lines in appropriate squares on the stick. The kit provides: 10 typing sticks incorporating a positive control, 0.125 ml peroxidase-labeled species-specific anti-mouse antibody, peroxidase substrate system comprising 1.0 ml hydrogen peroxide (30%) and ten 4-chloro-1-napthol tablets, protocol booklet and troubleshooting guide.

### EXAMPLES

This invention is further illustrated by the following examples which should not be construed as limiting. The teachings of all references, patents and published patent applications cited throughout this application, as well as the Figures are hereby incorporated by reference.

In the following examples, the hepatitis B virus surface antigen (HBsAg) was chosen as an illustrative antigen for the subject liposome-based vaccines. The antigen described in the following examples is a viral surface antigen, and is used for illustration only. These examples should be reasonably related to the result of using other antigens, such as the subject cancer antigens.

Hepatitis B infection is a significant health risk and is one of the leading causes of death worldwide. Antibody response against HBsAg is known to confer protection against live virus infection in humans, there are also plenty T cell epitopes available on this well characterized protein, thus it is a suitable model antigen to demonstrate immune responses in mice. In addition, recombinant hepatitis B virus protein and gene expression constructs are also commercially available, eliminating the requirement for costly reagent development. However, it should not be construed that the invention is limited to the HBsAg vaccine. Rather, the instant invention has broad uses in a wide-range of antigens, including the subject cancer antigens.

### Example 1

### Prime and Boost Immunization Protocol

To immunize a host animal such as CD1 or Balb/c mice, primary immunization using the subject HBsAg-liposome preparation was administered to the animal on week 0. At week 6, liposome-mediated secondary boost immunization was administered, optionally through a different route.

To optimize the vaccine delivery platform, we performed several experiments using different immunization strategies, as outlined below. In this series of experiments, liposomes were sized at 4 μm, a size reported to be effective in stimulating immune responses to protein antigens. Initial experiments tested the following parameters: dose of HBsAg-liposome preparations (3 μg or 15 μg/mouse), route of administration (oral, intranasal, or intramuscular), and physical form of the vaccine (fresh or lyophilized). Also tested was the effect of one (prime week 0) versus two (prime and boost weeks 0 and 6) rounds of immunization.

Samples were collected at the indicated time points for analysis of humoral and mucosal immune responses. For example, in certain experiments, the immunogenicity of the different preparations and administration protocols were measured by determining antibody responses in the serum at 2, 4, 6, 8, and 12 weeks after priming using HBsAg-specific ELISA assays for total serum Ig.

Negative controls included naïve mice, liposomes alone, and HBsAg protein alone. Animals were also immunized with either 3 μg GSK vaccine intramuscularly (Engerix™-B from GSK Biologicals, or “GSK HBsAg,” as positive control) or with 100 μg HBsAg-DNA vaccine intramuscularly to serve as benchmarks for Th2- and Th1-biased immune responses, respectively. Both outbred, genetically heterogenous CD1 mice (N=225 at 5/group) and inbred Th2-prone Balb/c mice (N=50 at 5/group) were immunized for comparison purposes. CD1 mice are an outbred strain (i.e., genetically heterogeneous) and are therefore representative of the diverse genetics found in humans.

For example, in one series of experiments, 10 mice were immunized at week by prime antigen. At week 6, a boost dose was administered to 5 of the 10 mice. Samples were collected (for example, from blood, feces, and vaginal wash) at weeks 2, 4, 6, 8, and 12 weeks for ELISA, ELISPOT, in vivo CTL assay, and Necropsy.

In a typical series of experiments, the following routes were used for various administrations: HBsAg-liposome (IN, IM, and oral); HBsAg-liposome+LT (oral, IN); liposome alone or HBsAg alone (IN, IM, oral); HBsAg-DNA vaccine or GSK vaccine (IM); no immunization control.

Examples below show representative data from these studies to demonstrate the optimal conditions for vaccination with the subject HBsAg-liposomes. The humoral immune response to the different vaccines was measured by determining HBsAg-specific total serum antibody responses and HBsAg-specific serum IgG antibodies using ELISA assays that we developed (supra). Although samples taken at 2, 4, 6, 8, and 12 weeks after priming were analyzed, only the responses at eight weeks after the first immunization are shown.

Most graphs include responses to the commercial GSK HBsAg vaccine as a positive control, and to serve as a benchmark that we wish to achieve for the test vaccines. No HBsAg-specific responses were detected in any naïve mice (data not shown). Closed symbols identify responses from mice that received one immunization, while cross-hatched, closed symbols (+) show responses from mice that received both a priming and a booster immunization. Results are presented as the dilution of sample tested (x-axis) versus the optical density at 450 nm in ELISA assays for total serum antibody (y-axis).

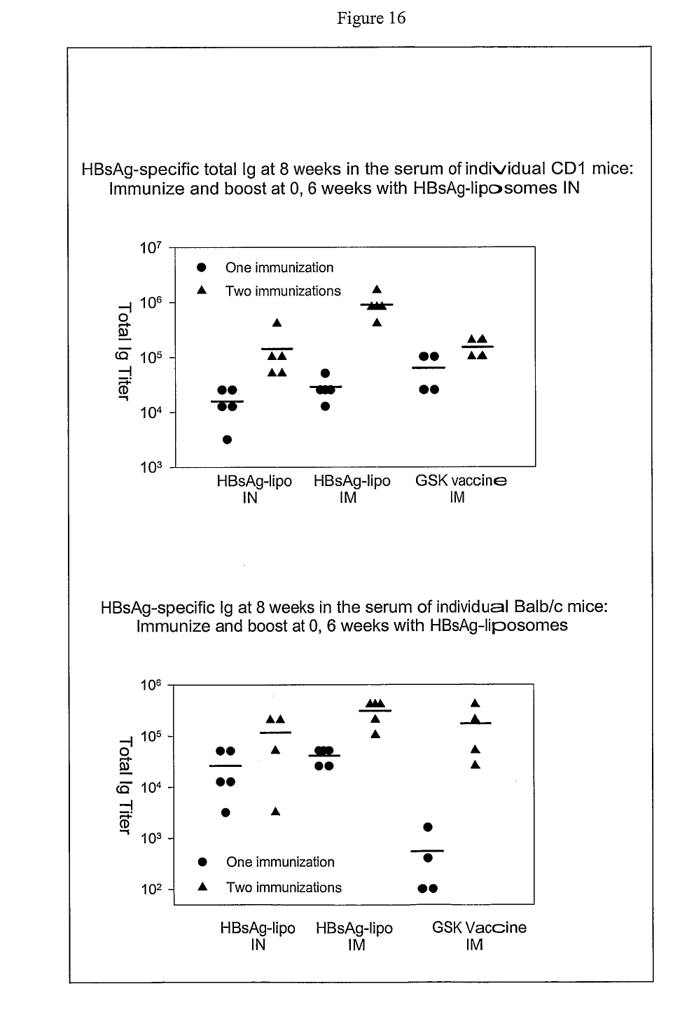

For clarity, most of the data presented here are averages from samples pooled from 5-10 mice per experiment (equal amounts of individual samples from groups of mice). FIG. 16 below is representative of the degree of variation that is observed between individual CD1 and Balb/c mice within an experiment. Individual responses to HBsAg-liposomes delivered intranasally or injected intramuscularly were compared to GSK vaccine injected intramuscularly.

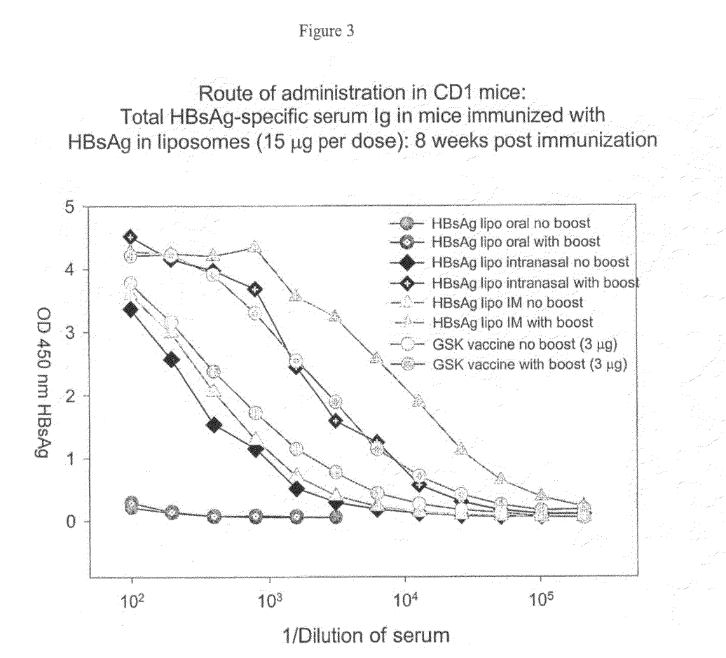

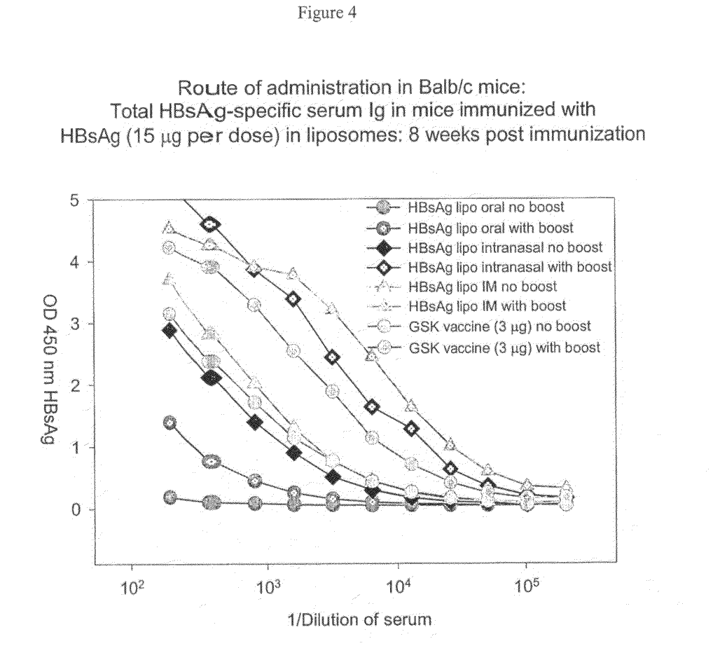

FIGS. 3 and 4 show a comparison between different routes of administration in CD1 and Balb/c mice, respectively. Applicants observed robust antibody responses following immunization with HBsAg-liposomes delivered intranasally (IN) or intramuscularly (IM). These responses were comparable to, or greater than, those observed with the commercial GSK HBsAg vaccine. Oral delivery of the same preparations was not effective in CD1 mice, and induced weak responses in Balb/c mice (2 out of 10 mice produced detectable antibody). All responding mice generated an enhanced HBsAg-specific antibody response after receiving a boost with the same dose and form of vaccine. A dose of 15 μg of HBsAg-liposome (shown) was more immunogenic that 3 μg per mouse for all routes of administration (data not shown). HBsAg alone induced antibody responses only when given intramuscularly, and liposomes not containing HBsAg were not immunogenic when given by oral, intranasal, or intramuscular routes (data not shown). Thus liposomes are a very effective adjuvant and delivery vehicle for the HBsAg antigen given by the IN or the IM routes.

### Example 2

### Analysis of Immune Responses to HBsAg

Two broad area of immune response in host animals were measured: humoral and cellular (T cell) response.

For humoral responses, antibody titers were measured from samples from serum and mucosal surfaces. Both HBsAg-specific antibodies and total Ig, Ig isotypes such as IgG, IgG1, IgG2a and IgA were measured.

For T cell responses, specific cytokine secretion (including IFN-gamma, IL-4), as well as in vivo cytolytic T cell (CTL) activity were measured according to standard protocol.

### Example 3

### Kinetics of HBsAg-specific serum Ig Levels in CD1 Mice after Intranasal (IN) Immunization with HBsAg Encapsulated in Liposome (HBsAg-Liposome)

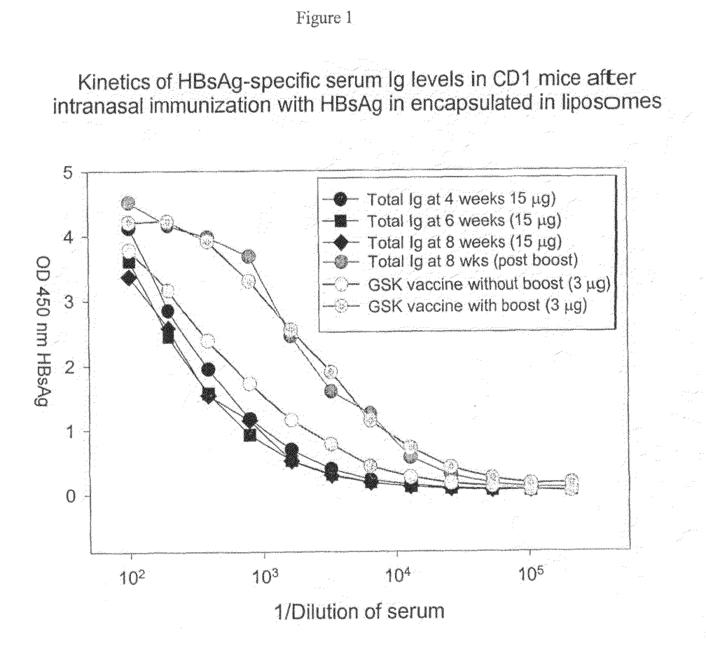

FIG. 1 illustrates the serum titer of HBsAg-specific antibodies after immunizing CD1 mice intranasally with 15 μg of HBsAg-liposome. The titers of various serum antibody samples were measured by standard ELISA assay using serial dilutions of serum samples (up to 105-106 fold dilution). The ELISA results expressed as arbitrary units were read at 450 nm as OD₄₅₀

The GSK vaccine at a dose of 3 μg was used as a positive control. There is a significant titer increase if a boost immunization using the same antigen was administered.

For HBsAg, 15 μg of antigen in liposome were used. Similarly, a significant boost in titer of HBsAg-specific antibody was observed if a second boost administration of the same Ag preparation was used. Without boost administration, however, titer reached its peak at about 4 weeks post the initial immunization, and stayed at similar levels through week 8. In addition, 15 μg of HBsAg-liposome is at least as effective as the commercially available GSK vaccine at 3 μg dosage.

### Example 4

### Dose Response to HBsAg-Liposome

Using a similar immunization protocol as in Example 3, CD1 mice were immunized, with or without a second boost administration, with either 3 μg or 15 μg of HBsAg encapsulated in the liposome preparation.

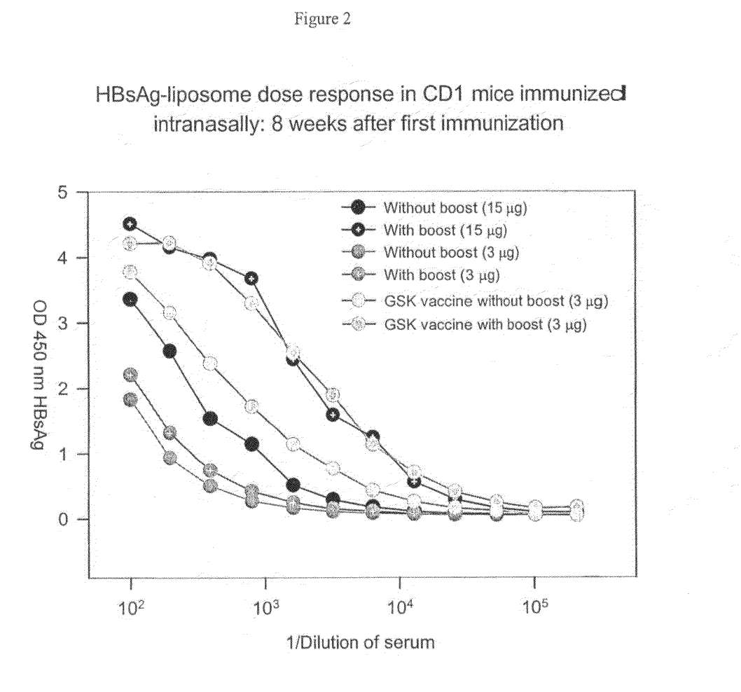

As Example 3 above, either with or without a second boost administration, 15 μg of HBsAg-liposome was at least as effective as the commercially available GSK vaccine (3 μg). However, 3 μg of HBsAg in the same liposome preparation was significantly less effective, and a second boost administration did not appear to result in a significant increase in titer. See FIG. 2.

### Example 5

### Routes of Administration

To test the effect of administration routes, CD1 mice and Balb/c mice were immunized with the same HBsAg-liposome preparation as described above, through oral, intranasal (IN) or intramuscular (IM) administration. Antibody titers were measured 8 weeks post (the initial) immunization.

It appeared that oral administration, with or without a second boost, did not elicit measurable immune response against the HBsAg (15 μg) at least in CD1 mice. The titer is only slightly higher in Balb/c mice receiving a second boost administration. These results indicate that oral administration is at best an ineffective route of administration for HBsAg-liposome (FIGS. 3 and 4).

However, in both CD1 and Balb/c, both IN and IM administration of 15 μg of HBsAg-liposome, with or without a second boost, were at least as effective as 3 μg of the GSK vaccine (FIGS. 3 and 4).

### Example 6

### Effects of Fresh or Lyophilized Liposome Preparations

To test whether the HBsAg-liposome preparation has to be used as fresh or lyophilized form, CD1 mice were immunized with either fresh or lyophilized liposome preparations, through different routes of administration. All serum Ig titers were measured 8 weeks post initial immunization.

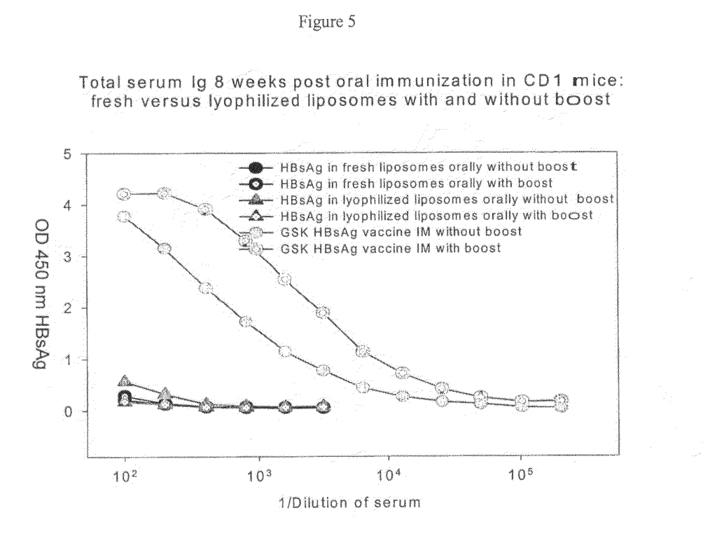

As shown in FIG. 5, oral administration is quite ineffective to elicit immune response in CD1 mice, no matter whether fresh or lyophilized liposome preparations were used, and no matter with or without a second boost administration.

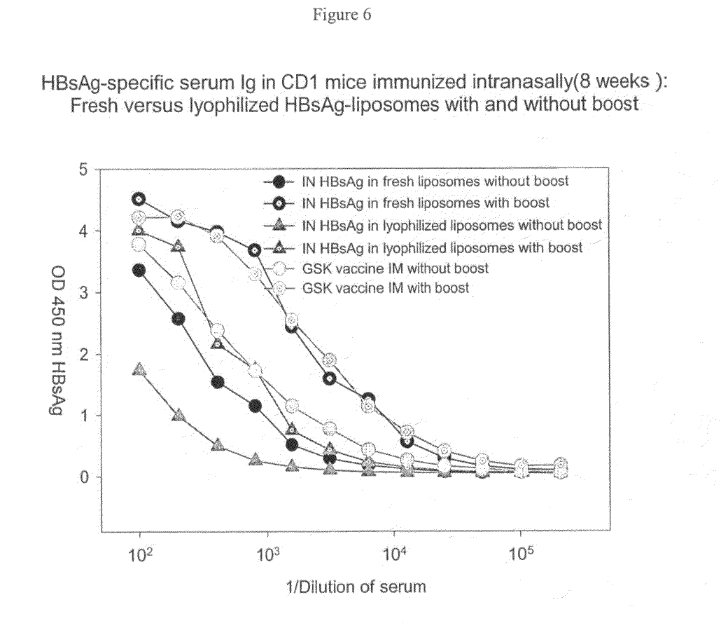

Intranasal administration of HBsAg-liposome, however, was at least as effective as the 3 μg GSK vaccine control, if the liposome preparation was fresh (FIG. 6). Comparable levels of titers were observed between the GSK vaccine and the instant HBsAg-liposome preparation, either with or without boost. But if lyophilized HBsAg-liposome preparation was used, the titer was only slightly reduced. In another separate experiment, however, lyophilized HBsAg-liposome preparation was slightly better that the fresh counterpart (Data not show) possibly due to batch variations.

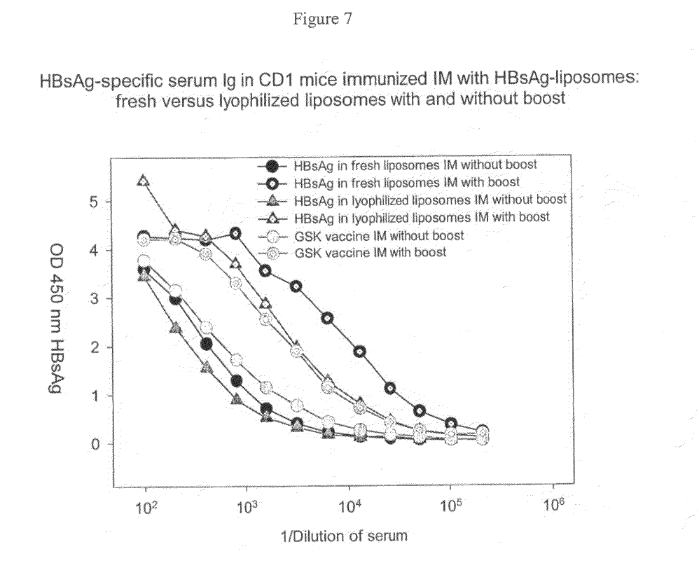

Similar result was obtained with 1M administration (FIG. 7). Both fresh and lyophilized HBsAg-liposome preparations had similar final titers as compared to each other, and to the GSK positive control.

These results indicate that reconstituted preparations of lyophilized HBsAg-liposomes were generally as effective as the fresh preparations when delivered by the IM (FIG. 5) or the IN routes (data not shown). The efficacy of the lyophilized preparations will simplify the distribution and storage of future vaccine formulations.

### Example 7

### Effects of LT Adjuvant

To determine whether serum antibody responses could be further enhanced to HBsAg-liposomes delivered intranasally or orally, Applicants co-administered this vaccine with the mucosal vaccine adjuvant LTR192G (LT). LT is a mutant form of heat-labile enterotoxin isolated from Escherichia coli, and demonstrates low toxicity in vivo. LT has been reported to enhance responses to intranasally delivered naked proteins.

Specifically, CD1 mice were immunized with 15 μg HBsAg in freshly prepared liposome preparation, administered orally or intranasally, either with or without second boost. In certain immunization protocol, LT adjuvant was added. AU serum Ig titers were measured 8 weeks post initial immunization.

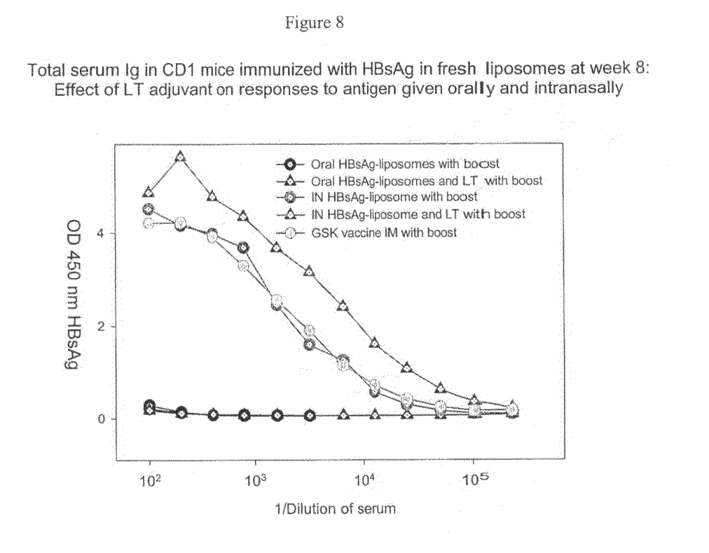

FIG. 8 indicated that when administered intranasally, LT adjuvant significantly increased the titer of HBsAg-specific antibodies, and this effect is additive to the second boost. However, when administered orally, LT adjuvant had no measurable effect on titer of anti-HBsAg antibody, since no measurable immune response was observed with oral administration. Compared to the serum anti-LT antibody level generated by IN delivery, LT alone as immunogen is much weaker in raising when administered orally (Data not show).

### Example 8

### Characterization of Polarized Th Responses

Cytokines are the hormonal messengers responsible for most of the biological effects in the immune system, such as cell mediated immunity and allergic type responses. Although they are numerous, cytokines can be functionally divided into two groups: those that are pro-inflammatory and those that are essentially anti-inflammatory but that promote allergic responses.

T lymphocytes are a major source of cytokines. These cells bear antigen specific receptors on their cell surface to allow recognition of foreign pathogens. They can also recognize normal tissue during episodes of autoimmune diseases. There are two main subsets of T lymphocytes, distinguished by the presence of cell surface molecules known as CD4 and CD8. T lymphocytes expressing CD4 are also known as helper T cells, and these are regarded as being the most prolific cytokine producers. This subset can be further subdivided into Th1 and Th2, and the cytokines they produce are known as Th1-type cytokines and Th2-type cytokines.

Th1-type cytokines tend to produce the pro-inflammatory responses responsible for killing intracellular parasites and for perpetuating autoimmune responses. Interferon gamma and interleukin-12 (IL-12) are the main Th1 cytokines. CTL activity is also high in a Th1-type immune response. In contrast, the Th2-type cytokines include interleukins 4, 5, and 13, which are associated with the promotion of IgE and eosinophilic responses in atropy, and also interleukin-10 (IL-10), which has more of an anti-inflammatory response. Th2 response provides help for the maturation of B cells to immunoglobulin-secreting cells, thereby primarily activating humoral defense mechanisms. CTL activity is generally low in a Th2-type immune response. In excess, Th2 responses will also counteract the Th1 mediated microbicidal action.

In general, the optimal scenario seems to be that humans should produce a well balanced Th1 and Th2 response, suited to the immune challenge. However, in reality, central to the concept of Th1 and Th2 subset generation is the tendency for these responses to become polarized. Thus, a Th1 or Th2 cytokine-producing profile will often dominate during an immune response by preferentially amplifying one Th subset and down-regulating the opposing response. This polarized response appears to be critical for host defense against many pathogenic organisms. Resistance to intracellular pathogens (such as virus) often requires a predominantly Th1 response, while Th2 responses are typically needed to fight extracellular parasites. Thus, T cell-derived cytokines produced by the host in response to an infectious agent determine the outcome of infection in many infectious disease models.

Whereas a pro-inflammatory Th1 response is usually required to control intracellular infections, there is also a need to balance the response (Taylor-Robinson, Int J Parasitol. 28(1): 135-48, 1998). It is important to produce a sufficiently potent type 1 response to keep the intracellular infection under control, while producing at the same time just enough of a type 2 or immunosuppressive response to prevent the protective response from causing damage to the host. The available data suggest that IFN-gamma, IL-12, and IL-10 cooperate to keep the Th2 response in check. Therefore, a successful outcome following an infection requires precise titration of Th1 and Th2 responses, appropriate to the type of infection. This is not just in terms of amount but also where, when and for how long these responses occur.

Several factors have been proposed, including the properties of antigens, dose of antigen, site of exposure and ongoing immune response in the host, to push a T-cell response towards a predominantly Th1 or Th2 phenotype. T helper cell responses to antigens may be characterized as polarized or as mixed. Polarized Th cell responses result from a skewing of the antigen-specific Th cell population towards a Th1 or a Th2 cytokine profile, and are reflected by antigen-specific IgG1:IgG2a ratios of <0.5 and >2.0, respectively. Mixed Th cell responses may contain both populations of T cells, and are reflected by antigen-specific IgG1:IgG2a ratios between 0.5 and 2.0. See Table B below.

TABLE B
Characteristics of Polarized Th1 and Th2 Responses

| Immune Response | Type 1 (Th1) | Type 2 (Th2) |
|---|---|---|
| Humoral Immunity | IgG1:IgG2a < 0.5 | IgG1:IgG2a > 2.0 |
| Cytokine Secretion | IFN-gamma, IL-12 (up); | IL-4, IL-10 (up); |
| | IL-4 (down). | IFN-gamma (down). |
| CTL Activity | High | Low |

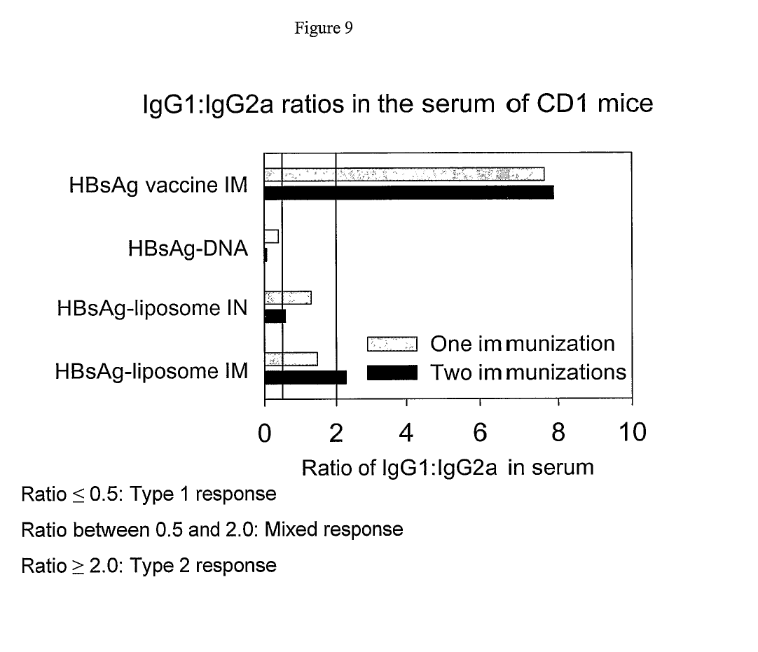

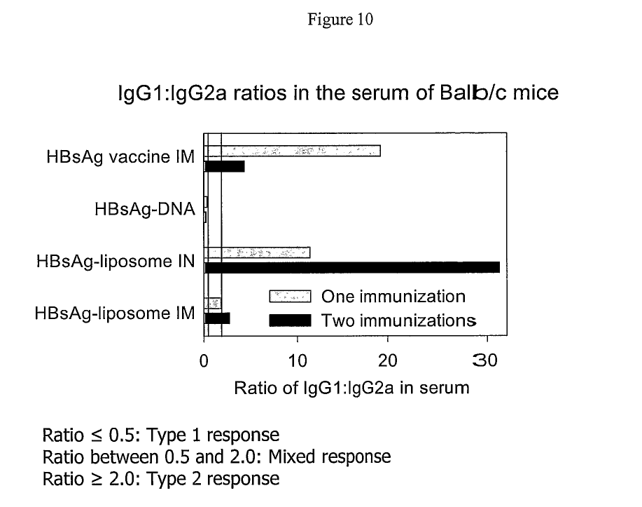

The nature of the immune responses to HBsAg-vaccine formulations was evaluated by determining IgG1:IgG2a ratios in the serum of CD1 and Balb/c mice at 8 weeks after the primary immunization. We designed quantitative ELISA assays to measure IgG1 and IgG2a antibodies specific for HBsAg. FIGS. 9 and 10 show the IgG1:IgG2a ratios in the sera of CD1 (FIG. 9) and Balb/c (FIG. 10) mice immunized by commercial GSK HBsAg vaccine, HBsAg-DNA vaccine, and HBsAg-liposome vaccine delivered intranasally or intramuscularly. Two vertical lines at 0.5 and at 2.0 in each panel demarcate three different patterns of antibody response (Th1, mixed or neutral, or Th2) in the regions from left to right.

In agreement with published results, the commercial HBsAg vaccine preferentially generated a Th2-type immune response in both the outbred CD1 and the inbred Balb/c strains of mice. As expected, the HBsAg-DNA vaccine generated a Th1-type immune response in both strains. Administration of HBsAg-liposomes intranasally generated a mixed response in CD1 mice and a Th2-biased response in Balb/c mice. In contrast, administration of HBsAg-liposomes intramuscularly generated a neutral immune response in both strains of mice.

A Th2 biased immune response is not always the most desirable response to vaccines since it may also be associated with the development of allergic responses to environmental agents. Therefore Applicants determined whether immune responses to HBsAg-liposomes could be modulated towards a more favorable mixed or Th1 response by including the LT adjuvant to the formulation.

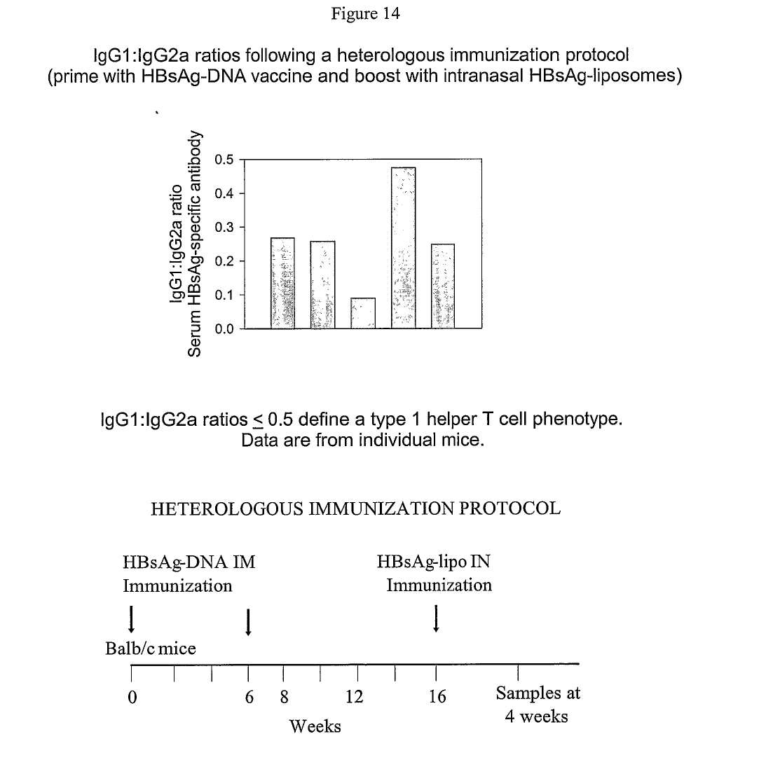

FIG. 14 below shows that co-administration of the LT mucosal adjuvant with HBsAg-liposomes intranasally to Balb/c and CD1 mice modified the nature of the immune response in both strains and generated Th1 polarized responses.

### Example 9

### Unique IgA response in Liposome-Mediated Immunization

Starting in the late 1960s, mucosal immunity was recognized as being an important defense against respiratory viruses when it was shown to correlate with protection against respiratory viruses in humans. At about the same time, the IgA class of antibodies was found to be especially prevalent in the respiratory tract and on other mucosal surfaces and to be the mediator of mucosal immunity. Other studies have since confirmed that mucosal immunity could ideally be stimulated in developing protection against infection through local vaccination.

Both IgG and IgA antibodies appear to be quite strain specific. With natural infection by respiratory viruses such as influenza virus, both a humoral and a systemic cellular immune response occur. The level of anti-viral antibody in the serum also correlates with protection.

One aspect of the instant invention provides a method to elicit mucosal immune responses by delivering HBsAg in liposomes directly to mucosal sites. An IgA-specific sandwich ELISA assay was designed to measure HBsAg-specific IgA in the serum and from mucosal sites. Serum, feces and vaginal washes from all of the experimental groups discussed above and some saliva samples were tested for IgA. End-point titers were established for each sample pool (N=4-5 mice per pool) by serially diluting test samples. End-point titers were defined as an OD at 450 nm in the IgA ELISA assay of greater than twice the value observed in samples from naïve non-immunized strain-matched mice at the same dilution of sample.

The only samples which showed detectable IgA responses were from mice that were given HBsAg-liposome vaccine by the intranasal route. Table C summarizes the results from the samples which scored positive in the HBsAg-specific IgA ELISA. IgA responses were detected in the serum and in most samples from mucosal sites after only one immunization. Boosting with a secondary immunization further increased IgA levels in most samples. Additionally, local immunization by the intranasal route generated secretory IgA responses at all remote mucosal sites sampled, including fecal pellets, vaginal washes, and saliva (data not shown for saliva due to the limited number of samples). These results demonstrate that immunization with HBsAg-liposomes by one route (intranasal) generated HBsAg specific mucosal IgA immunity at all of the mucosal sites sampled. The HBsAg-liposome vaccine, in contrast to the HBsAg protein or the liposomes alone, boosts serum immune Ig titers and also induces both serum and secretory IgA responses when administered intranasally.

All other immunogens fail to produce any detectable IgA responses (results not shown).

TABLE C
Summary of IgA responses in mice
immunized with HBsAg-liposomes

| | | Serum | Fecal | Vaginal | Saliva |
|---|---|---|---|---|---|
| Immunization | Strain | IgA | IgA | IgA | IgA |
| HBsAg-liposome | CD1 | Medium | Negative | ND§ | NA |
| IN (1×) | | | | | |
| HBsAg-liposome | CD1 | Medium | Low | ND§ | High |
| IN (2×) | | | | | |
| HBsAg-liposome | Balb/c | Low | Low | Medium | NA |
| IN (1×) | | | | | |
| HBsAg-liposome | Balb/c | High | High | High | High |
| IN (2×) | | | | | |
| HBsAg-liposome | CD1 | High | Negative | ND§ | Medium |
| IN (2×) + | | | | | |
| LT | | | | | |

§Not determined.

Samples of serum, feces, and vaginal washes were collected from five individual mice per group. Samples were processed individually as indicated in the Methods, and sample pools were made using equivalent volumes of sample per mouse. End-point titrations were determined for each sample and were defined as a serum titer >2× that observed for naïve unimmunized CD1 or Balb/c mice. The range of titer values that were used for the determination of the relative titer are shown below.

| Relative titer | Serum | Feces | Vaginal Wash |
|---|---|---|---|
| Negative | <100 | <100 | <25 |
| Low | 100-200 | 100-200 | 25-100 |
| Medium | 400-800 | 400-800 | 200-400 |
| High | >1600 | >1600 | 400-1600 |

§Not determined.

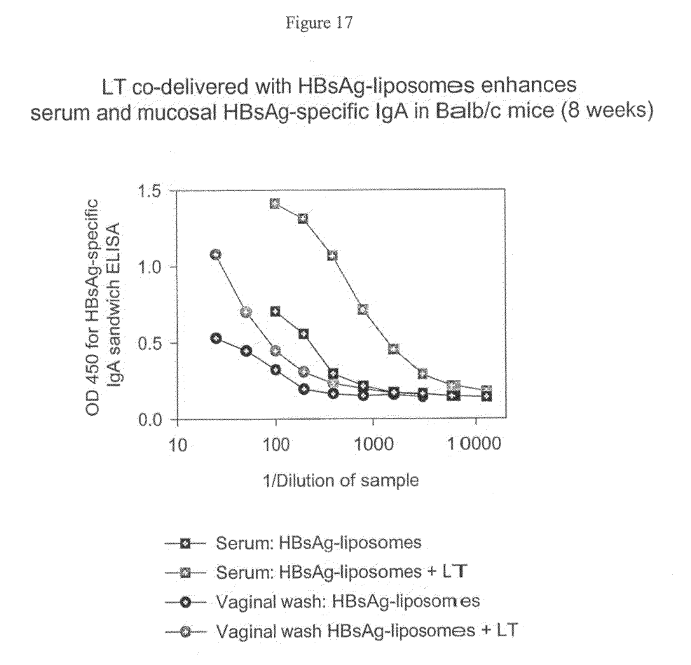

Table C also shows that co-administration of the mucosal adjuvant LT with HBsAg-liposomes further enhances serum IgA levels in CD1 mice. Enhanced serum and vaginal IgA responses were also observed in a second experiment using Balb/c mice (FIG. 17). The inclusion of LT in the HBsAg-liposome vaccine delivered intranasally therefore increases overall serum Ig responses (FIG. 5), biases the immune response towards a Th1 profile (FIG. 8), and enhances both serum and mucosal IgA responses to the HBsAg antigen (Table C and FIG. 9).

### Example 10

### Antibody Responses to HBsAg-DNA Vaccination is WEAK

DNA vaccine immunization has recently been described to generate a protective immune response in a host, including humans. See U.S. Pat. No. 6,632,663 and WO 03/075955 A1 (incorporated herein by reference).

To test the effectiveness of HBsAg-DNA as immunogen, CD1 mice and Balb/c mice were immunized intramuscularly with HBsAg-DNA, either with or without a second boost. Total HBsAg-specific serum Ig was measured 8 weeks post immunization as described before. GSK HBsAg vaccine was used as a positive control.

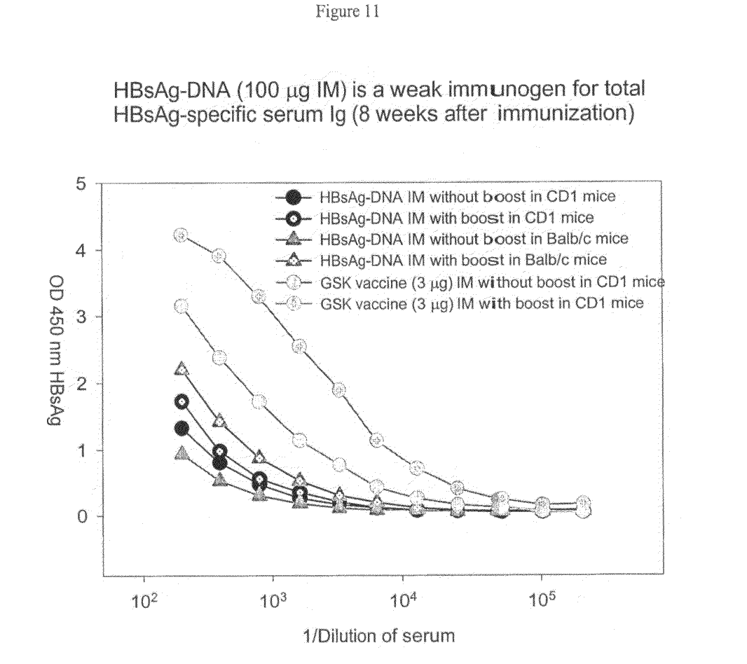

FIG. 11 indicated that, in contrast to HBsAg protein vaccines with adjuvants, intramuscular immunization with HBsAg-DNA produced a weak antibody response to HBsAg in both CD1 and Balb/c strains of rice. The elicited immune response, even with a boost, is weak to moderate at best when compared to the unboosted control GSK vaccine. However, Example 11 below reveals that HBsAg-DNA immunization effectively generated both T and B cell memory that could be mobilized with a low dose of intranasal HBsAg-liposome boost.

### Example 11

### Heterologous Immunization with HBsAg-DNA as Primer and HBsAg-liposome as Booster

Although intramuscular DNA immunization with HBsAg-liposomes was a weak stimulus for the production of total antibody (FIG. 11), DNA immunization may still be effective at establishing T cell memory with a favorable Th1 bias to the immune response. In this case existing T cell and B cell memory responses may be mobilized with an appropriate booster immunization. HBsAg-liposomes delivered intranasally may be an effective means to reveal T and B cell memory established by DNA immunization, and may additionally extend that memory to mucosal surfaces. Thus the invention provides a heterologous immunization experimental design as exemplified by the schematic below. Variations of the protocol are merely routine and are well-within the scope of invention.

In a representative protocol, mice were first immunized and primed with HBsAg-DNA vaccine intramuscularly, and then challenged with a low dose of HBsAg-liposome intranasally. In certain embodiments, the low dose is itself insufficient in eliciting an significant immune response when administered alone. When compared to a dose sufficient to elicit a full-scale immune response, the low dose is at most 50%, 40%, 30%, 20%, 10%, 5% or lower.

Balb/c mice were intramuscularly immunized by 100 μg of HBsAg-DNA at week 0, and again at week 6. At week 16, a boost of HBsAg-liposome was administered intramucosally at a reduced dose of 3 μg. Serum samples were obtained 2, 4, and 6 weeks following the boost. As a positive control, CD1 mice were immunized intranasally with 15 μg of HBsAg-liposome with a second boost. Positive control serum was obtained 8 weeks post initial immunization. As a negative control, serum from naïve CD1 mice were obtained at week 8.

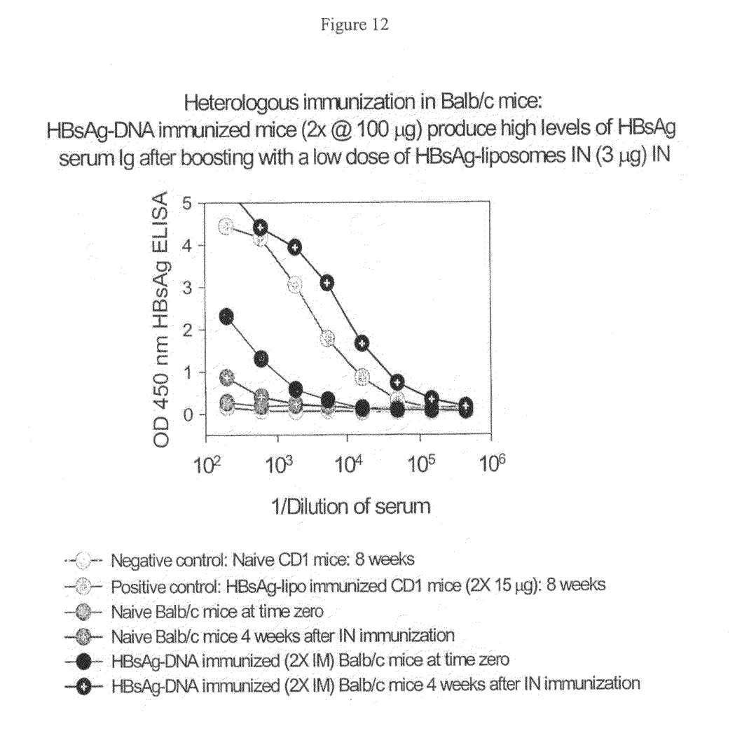

FIG. 12 shows results from this heterologous immunization protocol. Two rounds of IM HBsAg-DNA immunizations or one round of IN HBsAg-liposome (low dose) immunization alone induced weak serum antibody responses. However adding a second low dose boost with HBsAg-liposomes (3 μg) IN to the DNA immunization regimen markedly increased the total HBsAg-specific antibody response to a level even higher than seen in mice following two rounds of IN HBsAg-liposomes (high dose).

Quantitative ELISA assays were used to determine microgram amounts of HBsAg-specific IgG in the sera from this experiment. IgG levels after two rounds of DNA averaged 43.2 μg/ml, and increased to 248 μg/ml after boosting with HBsAg-liposomes. Administration of the liposome preparation alone generated only 0.6 μg/ml of serum IgG.

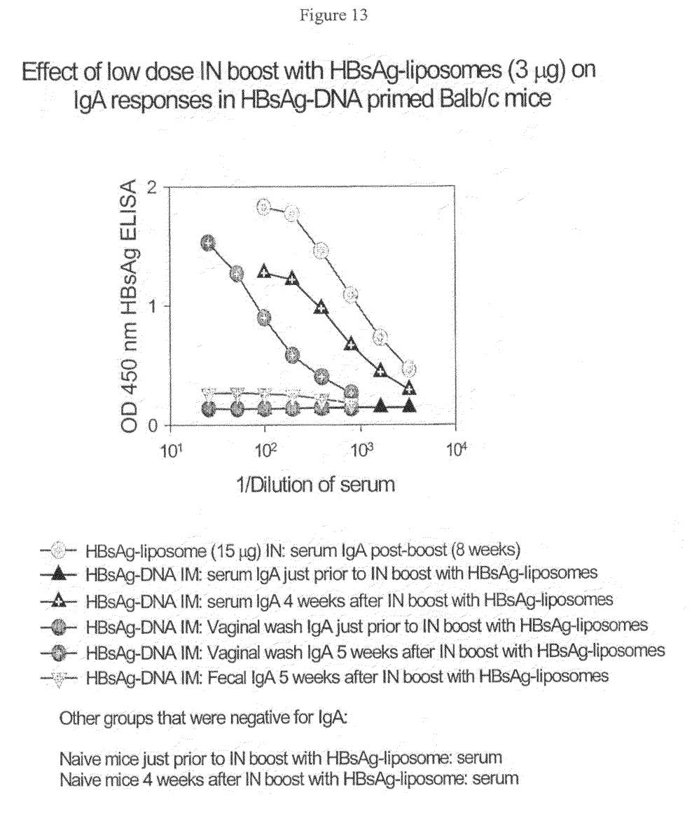

Remarkably, DNA immunization also primes the immune system for mucosal IgA responses, which also are mobilized following intranasal delivery of a low dose HBsAg-liposome boost. FIG. 13 shows that the DNA and liposome components of the heterologous immunization regimen alone fail to stimulate detectable IgA responses. However substantial IgA responses were induced in the serum and in the vagina using the heterologous DNA prime and HBsAg-liposome boost protocol. Secretory IgA was also induced in fecal samples using this protocol (data not shown). FIG. 13 also shows typical serum IgA responses following prime and boost with a higher dose of HBsAg-liposomes alone (15 μg HBsAg), which are a benchmark for robust IgA responses. Delivery of the HBsAg-liposome boost intranasally is far more effective at inducing serum and secretory IgA responses than delivery of the same preparation via the intramuscular route (data not shown).

The boost with HBsAg-liposomes must be delivered by the intranasal route in order to generate serum or mucosal IgA responses. If the boost is given IM rather than IN, only low level of secretory IgA is generated although circulating serum IgG responses are robust (data not shown).

Applicants also showed in FIGS. 3 and 4 that intranasally delivered HBsAg-liposomes prime mice for antibody responses as revealed by a subsequent 1N HBsAg-liposome boost. The priming established by intranasally delivered HBsAg-liposomes is also revealed by intramuscular boosting with HBsAg-DNA (data not shown).

Applicants also determined the IgG1:IgG2a ratio to evaluate the effect of the IN boost with HBsAg-liposomes on polarization of the T cell response (FIG. 14). Significantly, adding this boost to the DNA immunization protocol did not shift immune responses towards a Th2-type profile. Antibody responses 4 weeks after the second DNA immunization showed a Th1-type bias (IgG1:IgG2a ratio of 0.47). A Th1-biased response was still present after an intranasal boost with HBsAg-liposomes (IgG1:IgG2a ratio of 0.50). This experiment shows that DNA immunization establishes a strong and stable base of Th1-type memory which is effectively mobilized when HBsAg-liposomes are delivered intranasally.

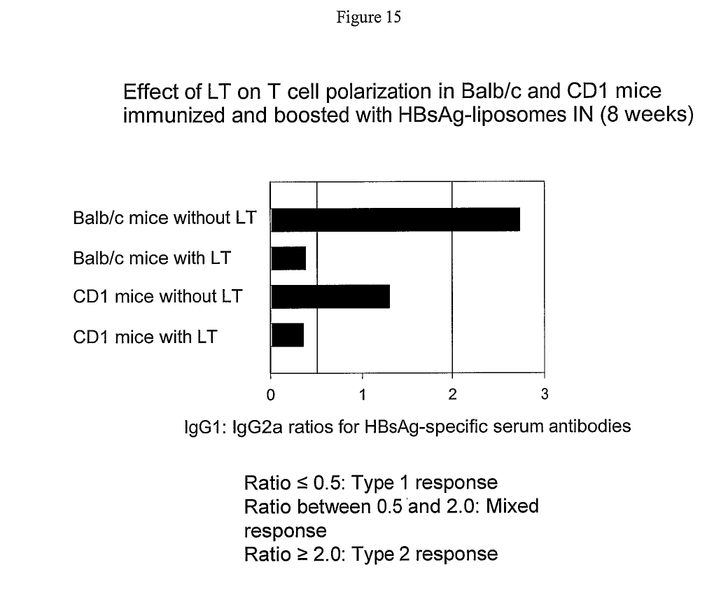

In addition, LT adjuvant tended to shift Th response to Th1 type response. FIG. 15 indicated that in Balb/c mice primed by HBsAg-DNA and boosted by low dose IN administration of HBsAg-liposome, an initial Th2 response was elicited. Addition of LT effectively shifts the response to Th1. In similarly immunized CD1 mice, an initial mixed-type response was effectively shifted to Th1 response if LT adjuvant was used.

The subject heterologous immunization protocols promote neutral or type 1 polarized Th responses and cell mediated immunity, while optionally retaining mucosal IgA responses (if the boost dose antigen-liposome preparation is administered intranasally). For protective immunity from intracellular pathogens, it is often desirable to initiate cell mediated immune responses, particularly for enhanced Th1 cytokine secretion and for the generation of CTLs. This is particularly useful for certain embodiments, where it may be desirable that an antigen and/or immunization protocol results in a Th1 response, rather than a Th2 or mixed-type response, since Th2 responses may be associated with certain undesirable side-effects similar to allergic reaction, especially in children.

In other embodiments, where the initial dose is the subject HBsAg-liposome preparation administered intranasally, and the boost dose is HBsAg-DNA administered intramuscularly, the resulting moderate immune response in the host animal tend to be a mixed-type, rather than a Th1 type response. This illustrates the contention that the order of the subject heterologous immunization is important in order to achieve the Th1 response.

### Example 12

### The Effect of Liposome Size on the Immune Responses to HBsAg-Liposomes

The effect of liposome size was evaluated by encapsulating HBsAg (15 μg/mouse) in liposomes sized at 4 μm, 1 μm and 0.2 μm. Quantitative ELISA assays were used to measure HBsAg-specific serum IgG levels after intranasal immunization with single sized liposomes or after intranasal immunization with an equal mixture of all three sizes. It is possible that a mixture of different sized liposomes may be more immunogenic than a single size of liposome. Total amounts of HBsAg and lipid were the same in the four groups.

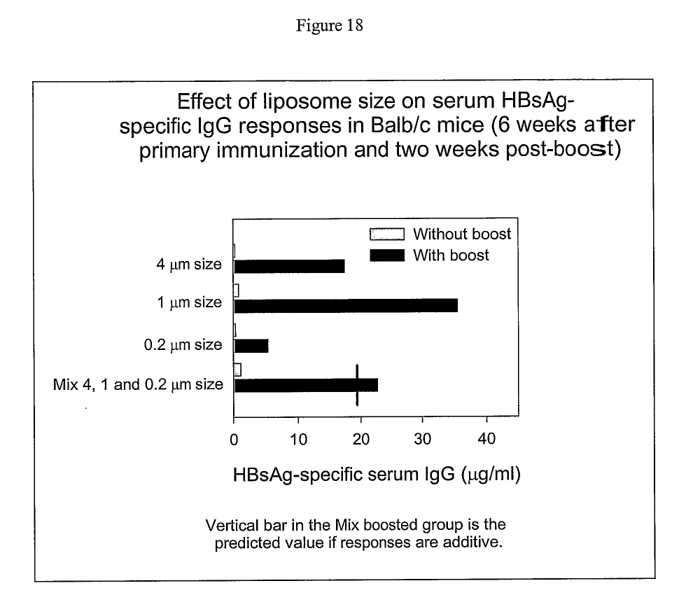

FIG. 18 shows that liposomes sized at 1 μm and 4 μm were most effective, while the smaller 0.2 μm liposomes were less effective at generating HBsAg-specific serum IgG. A mixture of equal parts of all three sizes generated an additive but not a synergistic response. The predicted response if the effects of the three sizes of liposomes combined is additive is shown on FIG. 18 as a vertical bar.

### Example 13

### The Effect of Liposome to Protein Ratio on the Immune Responses to HBsAg-Liposomes

We determined the importance of liposome to protein ratios in generating HBsAg-specific serum IgG antibodies by immunizing mice intranasally with HBsAg-liposomes (15 μg/mouse, primed at week 0 and boosted at week 6) at our standard content of liposomes, or at ½ and ⅓ of the liposome concentration. Reducing the liposome to HBsAg ratio by ½ and ⅓ reduced serum IgG responses disproportionately to 35% and 2% of the response obtained from our standard liposome preparation, respectively (n=8 animals per group; serum IgG measured on pools of sera from each group two weeks post-boost). The amount of liposome used to encapsulate the HBsAg therefore has a significant effect for generating high levels of HBsAg-specific serum antibody responses.

### Example 14

### CTL Activity and Cytokine Secretion in DNA Primed/HBsAg-Liposome Boosted Mice

Protective immunity from many intracellular pathogens often requires a Th1-biased immune response with high IFN-γ production, and the generation of cytolytic T lymphocytes (CTLs) which can kill pathogen infected cells. To evaluate the cell mediated immune responses generated by our vaccine delivery protocols, Applicants have established in vivo assays to measure CTL responses, and ELISPOT assays to enumerate frequencies of HBsAg-specific IFN-γ and IL-4 secreting cells (Th1 and Th2 responses, respectively).

In vivo CTL assays have been recently reported in the literature. Unlike conventional in vitro CTL assays, they provide a true measure of the ability of CTLs to kill targets in the in vivo setting. Applicants measured in vivo CTL activity in mice that received two rounds of HBsAg-DNA IM followed by a low dose 1N HBsAg-liposome boost. After the sample collection was completed for the measurement of antibody responses (six weeks or longer after the IN boost), mice received a DNA boost to mobilize the memory T cells into activated effector cells. 8-12 days later, the activity of mobilized CTL effector cells was quantified by determining their ability to specifically kill HBsAg peptide-pulsed targets that were adoptively transferred intravenously into the mice. The mice received a mixture of two populations of cells from naïve mice that were differentially labeled with the intravital dye CFSE (fluorescence emission in the FITC channel): CFSE^{low} cells that were not pulsed with peptide, and CFSE^{high} cells that were pulsed with the immunodominant peptide from HBsAg (amino acids 28-39 of HBsAg, IPQSLDSWWTSL-OH). The disappearance of the peptide-pulsed CFSE^{high} population relative to the unpulsed CFSE^{low} population is the measure of peptide-specific killing according to the formula shown below. The percentages of these two populations in cohorts of naïve recipients served as the internal control for cell engraftment. The percentage of CFSE^{low} and CFSE^{high} donor cells in the spleens of the recipients was determined by flow cytometric analyses. A summary of this assay is shown below.

### Target Cell Preparation:

Naïve splenocytes: CFSE^{low} labeled without peptide pulse.
Naïve splenocoytes: CFSE^{high} labeled with HBsAg peptide pulse.
Mix equal numbers of CFSE^{low} and CFSE^{high} cells (1-1.5×10⁷ cells of each).

### In Vivo CTL Assay:

Transfer target cells to cohorts of both immunized and naïve mice intravenously. 20-24 hours later, harvest spleens and process for flow cytometry and ELISPOT assays.

$$Ratio = Percentage of {CFSE}^{low } cells \text{:} {CFSE}^{high} cells$$

$$\% Specific Lysis = 1 - \underline{ratio in naïve mice} \times 100$$

$$ratio in immunized mice$$

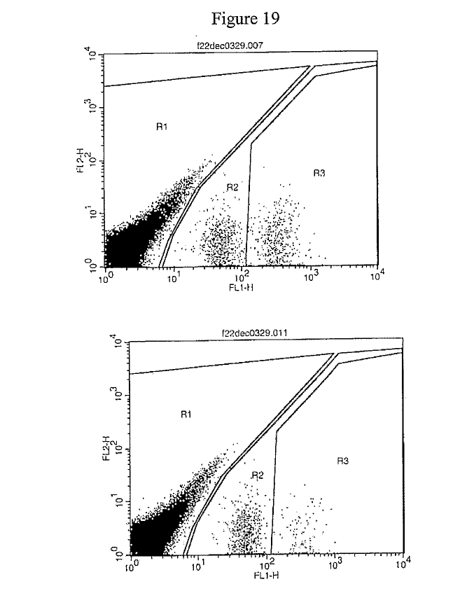

Representative flow cytometric analyses are shown in FIG. 19 for one of our assays. The left panel shows CFSE^{low} (R2 on the graph) and CFSE^{high} (R3 on the graph) populations of donor cells that were recovered from the spleens of naïve mice 22 hours after injection of the CFSE labeled target cells. CFSE fluorescence is shown in the FITC or FL1 channel on the x-axis. FL1 fluorescence is displayed versus irrelevant FL2 fluorescence solely for the purpose of excluding auto-fluorescing cells from the analysis. The right panel shows the same analysis from mice that had received the heterologous immunization protocol. The disappearance of CFSE^{high} (specific peptide pulsed) population from the R3 region indicates specific killing by CTLs.

High levels of HBsAg peptide-specific CTL killing activity were observed in Balb/c mice that were primed intramuscularly with HBsAg-DNA and boosted intranasally with HBsAg-liposomes. In vivo CTL activity averaged 78% (range of 65-91% specific killing) in a grouping of four mice.

Spleen cells from mice that received the heterologous immunization regimen were also cultured in vitro for the measurement of IFN-γ and IL-4 secreting cells by ELISPOT assays. The ELISPOT assays were established using commercial antibody pairs and reagents (BD Pharmingen) and Multiscreen-IP multiwell ELISPOT plates (Millipore, Hopkington, Mass.). In vitro cultures were stimulated with HBsAg peptide at a concentration of 10 μg per 2.5×10⁶ cells per ml for 20-22 hours. Cultures with no peptide were used as a negative control, and cultures stimulated with anti-CD3 or Con A were used as positive controls.

Applicants observed high frequencies of HBsAg-specific IFN-γ secreting cells, but only very low frequencies HBsAg-specific IL-4 secreting cells in mice immunized with the heterologous immunization protocol. These results directly confirm our conclusion derived from the analysis of serum IgG1: IgG2a ratios, that the heterologous immunization protocol generates a Th1-type cytokine biased immune response. IFN-γ and IL-4 ELISPOT plates from this study are being quantitatively analyzed by Zellnet Consulting Inc. (New York, N.Y.) for both frequencies of spots and spot size using a digital plate reader and software designed for this purpose.

Further analysis using immunized mice from these experiments for in vivo CTL activity and frequencies of IFN-γ and IL-4 secreting cells, measurement of in vivo CTL activity at mucosal sites (nasal passages, lung and vagina) by the use of digital fluorescence imaging on frozen tissue sections from the mice are not shown.

Table D below summarizes the characteristics of the immune responses that were generated with specific HBsAg vaccine delivery platforms. Using HBsAg as a model antigen, Applicants have shown that immune responses can be directed towards specific outcomes (i.e., antibody response levels, T helper cytokine profiles (Th1 v. Th2), development of secretory IgA mucosal immunity, and elicitation of cytolytic T cells). These studies provide a rational basis that will can be used to tailor immune responses to generate protective immunity against specific pathogens under specific circumstances to favor Th1 or Th2 type immune responses, or a mixed/more balanced response.

TABLE D
Summary of the characterization of immune responses to HBsAg vaccines

| Vaccine delivery platforms | | | | Mucosal | |
|---|---|---|---|---|---|
| Prime | Boost | Adjuvant | Th profile | Immunity | CTLs^{§} |
| GSK vaccine IM | GSK IM | Alum | Th2 | None | (None) |
| DNA vaccine IM | DNA IM | None | Th1 | None | (Medium) |
| HBsAg-lipo IM | HBsAg-lipo M | Liposome | Mixed | None | (Medium) |
| HBsAg-lipo IN | HBBsAg-lipo IN | Liposome | Mixed to Th2 | High | (Medium) |
| H-lipo + LT IN | H-lipo + LT IN | Liposome, LT | Th1 | High | (Medium) |
| DNA vaccine IM | HBsAg-lipo IM | Liposome | Th1 | Low | (High) |
| DNA vaccine IM | HBsAg-lipo IN | Liposome | Th1 | High | High |
| HBsAg-lipo EN | DNA IM | Liposome | Mixed to Th2 | (Low) | (Medium) |

^{§}Levels of CTLs and mucosal immunity that are shown in parentheses are the predicted levels. Experiments for these groups and for ELISPOT determinations of the frequencies of HBsAg-specific IFN-γ and IL-4 secreting cells are in progress.

In summary, the results show that specific doses of HBsAg, when encapsulated in liposomes of a certain composition and size (such as 4 μm size average), are potent vaccines for the production of serum antibody. Robust responses are observed when the vaccine is administered intranasally or intramuscularly. In contrast, HBsAg-DNA injected intramuscularly is less effective in inducing HBsAg-specific serum antibody. Applicants did observe that the mice tolerated the intranasal vaccines in all formulations without evidence of distress or mortality.

Only intranasal immunization with HBsAg-liposome generated systemic and mucosal IgA responses, while all other immunization regimens failed to produce IgA response. And finally, heterologous immunization regimen with HBsAg-DNA vaccine by IM, followed by HBsAg-liposome by IN is an ideal vaccine delivery platform, in that it produces synergistic serum antibody response, robust mucosal and serum IgA response, and potentially enhanced antigen-specific T-cell response and IFN-gamma secretion without changing the Th1-type response established by DNA vaccine priming.

The biodistribution of HBsAg-liposomes was not measured directly. However the ability of vaccine formulations to stimulate local immunity at mucosal surfaces was determined and the results presented above.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

A skilled artisan will recognize, or be able to ascertain using no more than routine experimentation, many equivalents to the specific embodiments of the invention described herein. Such equivalents are intended to be encompassed by the following claims.

1. A method for eliciting an immune response to a cancer cell surface antigen in an individual with cancer, comprising
administering to the individual an antigen-liposome preparation in sufficient dose to elicit the immune response to the cancer cell surface antigen,
wherein the antigen-liposome preparation comprises the cancer cell surface antigen and a liposome, and wherein the immune response is sufficient for treating the cancer.

2. The method of claim 1, wherein the cancer cell surface antigen is expressed only in cancer cells in the individual.

3. The method of claim 1, wherein the cancer cell surface antigen is expressed only by
(a) cancer cells, and
(b) non-cancer cells normally located in one or more immune-privileged sites or tissues in the individual, wherein the one or more immune-privileged sites of the individual are selected from the group consisting of: brain, spinal cord, anterior chamber of the eye, vitreous cavity and subretinal space of the eve, adrenal cortex, and a reproductive organ selected from the group consisting of: testis, ovary, and uterus.

4. (canceled)

5. The method of claim 3, wherein the cancer cell surface antigen is a neuropeptide, a neuroendocrine peptide, or a neuroendocrine peptide receptor.

6. The method of claim 3, wherein the cancer cells are from small cell lung cancer, breast cancer, prostate cancer, or neuroendocrine tumor.

7. The method of claim 6, wherein the neuroendocrine tumor is a carcinoid tumor; an adrenal pheochromocytoma; a gastrinoma (causing Zollinger-Ellison syndrome); a glucagonoma; an insulinoma; a medullary carcinoma of the thyroid; a multiple endocrine neoplasia syndrome; a pancreatic endocrine tumor; a paraganglioma; or a VIPoma (vasoactive intestinal polypeptide tumor).

8. The method of claim 7, wherein the carcinoid tumor is found in gastrointestinal tract, lung, intestinal tract, appendix, rectum, bronchial tubes, or ovary.

9. The method of claim 3, wherein the cell surface antigen is selected from the group consisting of: alpha fetal protein (AFP), BLP25 vaccine or MUC1 mucin, STn or STn-KLH, GD3 or its anti-idiotypic antibody BEC2, and VEGFR2.

10. The method of claim 1, wherein the individual is a human or non-human mammal.

11-14. (canceled)

15. The method of claim 1, wherein the antigen-liposome preparation is administered intranasally, intramuscularly, subcutaneously, intravenously, or orally to the individual.

16-20. (canceled)

21. The method of claim 1, wherein the antigen-liposome preparation is administered with at least one vaccine adjuvant, such as LT R192G.

22. The method of claim 1, further comprising measuring humoral and/or cellular immune responses to the cancer cell surface antigen.

23. The method of claim 22, wherein the humoral immune response includes total antibody (Ig) titers in serum or at mucosal surfaces; titers of anti-HBsAg-specific antibodies in serum or at mucosal surfaces; titers of specific antibody isotypes and/or sub-types including IgG, IgA, IgG1, and IgG2a; ratio of IgG1 and IgG2a.

24. The method of claim 22, wherein the cellular immune response includes in vivo cytotoxic T cell (CTL) activity; secretion of cytokines characteristic of Th1 response including IL-12 and IFN-gamma; secretion of cytokines characteristic of Th2 response including IL-4, IL-5, IL-10, and IL-13; and T-helper cell profile (e.g., Th1 versus Th2 response).

25. The method of claim 22, wherein the humoral and/or cellular immune responses are measured from samples obtained from the individual about 2, 4, 6, or 8 weeks post the last boost, or about 8 weeks post the initial administration.

26. A method for selectively killing cancer cells expressing a cancer cell surface antigen in an individual in need thereof, comprising
administering to the individual an antigen-liposome preparation, under conditions that result in production in the individual of antibodies against the cancer cell surface antigen,
wherein the antibodies produced bind the cancer cell surface antigen on cancer cells in the individual, thereby killing cancer cells that express the cancer cell surface antigen.

27. The method of claim 26, wherein the cancer cell surface antigen is expressed only in cancer cells in the individual.

28. The method of claim 26, wherein the cancer cell surface antigen is expressed only by (a) cancer cells, and (b) non-cancer cells normally located in one or more immune-privileged sites or tissues in the individual, wherein the one or more immune-privileged sites of the individual are selected from the group consisting of: brain, spinal cord, anterior chamber of the eye, vitreous cavity and subretinal space of the eye, adrenal cortex, and a reproductive organ selected from the group consisting of: testis, ovary, and uterus.

29. (canceled)

30. The method of claim 28, wherein the cancer cell surface antigen is a neuropeptide, a neuroendocrine peptide, or a neuroendocrine peptide receptor, and wherein the cancer cells are small cell lung cancer cells, breast cancer cells, or prostate cancer cells.

31-34. (canceled)

35. A method of treating a cancer in an individual in need thereof, while protecting non-cancer cells of the individual from the adverse effects of the treatment, comprising
administering to the individual a therapeutically effective amount of a composition comprising a cancer cell surface antigen and a liposome, under conditions that result in production of antibodies against the cancer cell surface antigen,
wherein the antibodies bind the cancer cell surface antigen on cancer cells in the individual, and wherein the cancer cell surface antigen is expressed only in cancer cells and in non-cancer cells normally located in one or more immune-privileged sites or tissues of the individual, wherein the one or more immune-privileged sites of the individual are selected from the group consisting of: brain, spinal cord, anterior chamber of the eye, vitreous cavity and subretinal space of the eye, adrenal cortex, and a reproductive organ selected from the group consisting of: testis, ovary, and uterus.

36. (canceled)

37. The method of claim 35, wherein the cancer cell surface antigen is a neuropeptide, a neuroendocrine peptide, or a neuroendocrine peptide receptor, and the cancer cells are small cell lung cancer cells, breast cancer cells, or prostate cancer cells.

38-41. (canceled)
